(19) United States
(12) Patent Application Publication
(10) Pub. No.: US 2011/0064745 A1
(43) Pub. Date: Mar. 17, 2011

(54) Therapeutic Antibodies, Antibody Fragments And Antibody Conjugates

(76) Inventors: Michael F. Good, The Gap (AU); Michael R. Batzloff, Durack Queensland (AU); Manisha Pandey, Pakinson Queensland (AU)

(21) Appl. No.: 12/861,228

(22) Filed: Aug. 23, 2010

Related U.S. Application Data
(63) Continuation of application No. 11/950,217, filed on Dec. 4, 2007, now abandoned.
(60) Provisional application No. 60/868,447, filed on Dec. 4, 2006.

(51) Int. Cl. A61K 39/395; C07K 16/12; A61P 31/04; A61P 37/04

(52) U.S. Cl. 424/165.1; 530/389.5; 530/391.1; 530/388.4; 530/387.3

(57) ABSTRACT

The present invention provides compositions, including pharmaceutical compositions, comprising an amount of an antibody, antibody fragment or antibody conjugate sufficient to treat Group A streptococcus (GAS) infection or complication thereof in a subject or a disease or complication associated with GAS infection in a subject wherein said antibody, antibody fragment or antibody conjugate binds immunospecifically to a B-cell epitope of GAS M-protein. The present invention also provides methods for the prophylactic or therapeutic treatment of infection by GAS and complications thereof comprising administering the compositions to a subject in need thereof.

### CROSS-REFERENCE TO RELATED APPLICATION

This application claims priority under 35 USC §119 to U.S. Ser. No. 60/868,447 filed Dec. 4, 2006 the contents of which are hereby incorporated in their entirety.

### FIELD OF THE INVENTION

The present invention relates to antibody therapeutics for infectious diseases, for example, employing antibodies, antibody fragments and antibody conjugates that immunospecifically bind to a B-cell epitope of M-protein of Streptococcus pyogenes or Group A streptococci (herein “GAS”). The invention also provides compositions comprising said antibodies and/or antibody fragments and/or antibody conjugates, and methods for preventing, treating or ameliorating symptoms associated with GAS infection utilizing such compositions.

### BACKGROUND OF THE INVENTION

### General

The following publications provide conventional techniques of molecular biology, microbiology, virology, recombinant DNA technology, peptide synthesis in solution, solid phase peptide synthesis, and immunology. Such procedures are described, for example, in the following texts that are incorporated by reference:
Sambrook, Fritsch & Maniatis, Molecular Cloning: A Laboratory Manual, Cold Spring Harbor Laboratories, New York, Second Edition (1989), whole of Vols I, II, and III;
DNA Cloning: A Practical Approach, Vols. I and II (D. N. Glover, ed., 1985), IRL Press, Oxford, whole of text;
Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
Nucleic Acid Hybridization: A Practical Approach (B. D. Hames & S. J. Higgins, eds., 1985) IRL Press, Oxford, whole of text;
Animal Cell Culture: Practical Approach, Third Edition (John R. W. Masters, ed., 2000), ISBN 0199637970, whole of text;
Immobilized Cells and Enzymes: A Practical Approach (1986) IRL Press, Oxford, whole of text;
Perbal, B., A Practical Guide to Molecular Cloning (1984);
Methods In Enzymology (S. Colowick and N. Kaplan, eds., Academic Press, Inc.), whole of series;
J. F. Ramalho Ortigão, “The Chemistry of Peptide Synthesis” In: Knowledge database of Access to Virtual Laboratory website (Interactiva, Germany);
Handbook of Experimental Immunology, Vols. I-IV (D. M. Weir and C. C. Blackwell, eds., 1986, Blackwell Scientific Publications).
Geysen, H. M., Rodda, S. J., Mason, T. J., Tribbick, G., Schoofs, P. G. (1987) “Strategies for epitope analysis using peptide-synthesis.” Journal of Immunological Methods 102: 259-274; and
Kohler and Milstein, in Nature 256:495-497 (1975)
Kohler & Milstein, Eur. J. Immunol. 6:511-519 (1976)
Harlow, E., Lane, D. (1988). Antibodies: A Laboratory Manual. Cold Spring Harbour, Cold Spring Harbour Laboratory.
Huse, et al., Science 246:1275-1281 (1989).
Campbell “Monoclonal Antibody Technology, The Production and Characterization of Rodent and Human Hybridomas” in Burdon et al., (Eds.), Laboratory Techniques in Biochemistry and Molecular Biology, Volume 13, Elsevier Science Publishers, Amsterdam (1985);
Birnbaum, S.; Uden, C.; Magnusson, C. G. M.; Nilsson, S. Analytical Biochemistry 206 (1992) 168-171.
Coligan, J. E. et al. (Eds.) Current Protocols in Immunology, Wiley Intersciences, N.Y., (1999).
Lou, S. C.; Patel, C.; Ching, S.; Gordon, J. Clinical Chemistry 39 (1993) 619-624).

### Description of Related Art

Streptococcus pyogenes, also known as Group A Streptococcus (GAS), is a serious human pathogen capable of causing a variety of human diseases ranging from uncomplicated pharyngitis and pyoderma to severe life threatening invasive infections. Infections caused by GAS range from uncomplicated skin and soft tissue infections to life threatening invasive diseases such as bacteremia and necrotizing fasciitis as well as non-supperative sequalae such as rheumatic fever, rheumatic heart disease and acute glomerulonephritis.

Infections due to GAS represent a public health problem of major proportions in both developing and developed countries. Illness attributable to GAS infection results in a huge burden to health care systems worldwide, as there are an estimated over 25-35 million infections per year in the US alone. Although uncomplicated pharyngitis and skin and soft-tissue infections account for most of these infections, there is a resurgence in the incidence of the life-threatening illnesses, such as necrotizing fasciitis and toxic shock syndrome, in hospitals and other institutions. Uncomplicated infection can also lead to serious sequelae, such as, acute rheumatic fever (ARF) and glomerulonephritis. Acute rheumatic fever continues to be a leading cause of heart disease worldwide. WHO estimates that GAS causes 517,000 deaths worldwide annually, with approximately one-third of those deaths (163,000) related to invasive GAS disease and the remainder (354,000) related to nonsuppurative sequelae of GAS infections (Bisno A L, et al., Clin Infect Dis 2005 41(8):1150-1156).

Presently, penicillin is still used as a first-line therapy in the treatment of most GAS infections. Currently available methods of prevention are either inadequate or ineffective, as evidenced by the morbidity and mortality still associated with this pathogen worldwide. The current situation with respect to the health care burden and the treatment and prevention of GAS infections therefore warrants the development of other preventative/therapeutic treatment measures.

The surface M protein is the major virulence determinant and protective antigen of GAS. In the immune host, M protein antibodies are opsonic and promote ingestion and killing of GAS by phagocytic cells. The M proteins of GAS isolates are multivalent and may elicit the production of antibodies that cross-react with human tissues.

A non-host reactive, conformationally constrained, minimal B cell epitope of the GAS M protein has been identified, which comprises the sequence RDLDASREAK (SEQ ID NO: 1) (International Patent Publication No. WO 96/11944; Heyman W A, et al., Int Immunology 1997 8(11):1723-1733). In particular, WO 96/11944 disclosed ten chimeric peptides designated J1-J9 and J14 (FIG. 1, herein and SEQ ID NOs: 2-11)) derived from a peptide designated p145, which has the sequence LRRDLDASREAKKQVEKALE (SEQ ID NO: 12). To construct nine of these chimeric peptides i.e., J149, different 12-mer peptides derived from the p145 sequence were embedded within a heptad repeat sequence VKQLEDK (SEQ ID NO: 13; designated as the “(GCN4)₄ framework”) having a similar native conformation to the M protein epitope. To construct chimeric peptide J14, a 14-mer peptide derived from p145 was embedded within a heptad repeat sequence. Conservative amino acid substitutions were also incorporated into the peptides J1-J9 and J14 wherever an identical residue was found in both the heptad repeat sequence and the p145 sequence. The peptides were conjugated to diphtheria toxoid (DT) by glutaraldehyde fixation and used to immunize mice against GAS. Vaccines based on the chimeric peptide immunogens described in WO 96/11944 show much promise.

Poor vaccination uptake, the on-going health risk for non-immunized individuals infected with GAS, and the possible ill-health in immunized individuals in the early phase of a sever acute GAS infection e.g., before an immune response is mounted, make other therapeutic and prophylactic approaches for the treatment of GAS infection desirable. Such therapies may be adjunct to other treatments such as vaccination or antibiotics, or stand-alone therapies.

### SUMMARY OF THE INVENTION

In work leading up to the present invention, the present inventors sought to attain a better understanding of the mechanism of protection achieved by M protein-based vaccines and, in particular, to elucidate the role of antibodies in protection and to determine whether or not there was a role for T-cells in protection.

As exemplified herein for a murine model of GAS infection, administration of antisera or purified IgG against a GAS M protein B-cell epitope to naïve recipient mice protects against a subsequent GAS challenge. Such treatment produced antibody levels in naïve mice approximately the same as those in donor animals. The level of protection conferred by passive transfer of antibodies was about the same as for vaccination with a peptide vaccine comprising the B-cell epitope. These data indicate the efficacy of antibody therapy for the treatment of GAS infection, especially severe acute GAS infection.

In a longitudinal study, vaccinated animals responded better in the long term than animals receiving antisera, and the decline in efficacy of antibody therapy correlates with decreasing serum antibody levels over time. These data suggest that, for long-term treatment, e.g., of non-vaccinated or immune-compromized subjects undergoing a severe acute GAS infection, it is preferred to maintain the level of serum antibodies such as by multiple administrations or providing a single initial dose of high antibody titre.

The present inventors have also shown that administration of antisera against GAS M protein reduces symptoms of a GAS infection in a subject previously infected with GAS. The level of protection conferred by passive transfer of antibodies was about the same as observed in animals vaccinated with a peptide vaccine comprising the B-cell epitope prior to infection. These data indicate the efficacy of therapeutic antibody treatment of GAS infection, especially severe acute GAS infection.

As exemplified herein, the inventors have also shown that passive administration of antisera against GAS M protein reduces symptoms of a GAS infection in a subject that lacks B cells and T cells and that has been previously infected with GAS. These data indicate the efficacy of therapeutic antibody treatment in immunosuppressed or immunocompromised subjects suffering from a GAS infection.

### Specific Embodiments

The scope of the invention will be apparent from the claims as filed with the application that follow the examples. The claims as filed with the application are hereby incorporated into the description. The scope of the invention will also be apparent from the following description of specific embodiments and/or detailed description of preferred embodiments.

The present invention provides a composition comprising an amount of an antibody as defined herein or as described according to any embodiment hereof, including an antibody fragment or antibody conjugate, sufficient to treat GAS infection or complication thereof in a subject or a disease or complication associated with GAS infection in a subject wherein said antibody or antibody fragment or antibody conjugate binds immunospecifically to a B-cell epitope of Group A streptococci (GAS) M-protein.

As used herein, the term “amount” refers to a concentration of antibody or antibody fragment or antibody conjugate as determined by any means known to a skilled artisan, including antibody titre of a unit dose, or a concentration of a unit dose or multiple dose of an antibody or antibody fragment or antibody conjugate. Preferably, an amount of an antibody or antibody fragment or antibody conjugate sufficient to treat or prevent GAS infection or complication thereof in a subject or a diseases associated with GAS infection is achieved using a high titre antibody or antibody fragment, such as, for example a titre sufficient to neutralize GAS in an infected subject. By “neutralize” is meant that the antibody or antibody fragment or antibody conjugate blocks the infective capacity of GAS and/or toxicity of a GAS toxin. The precise amount of the antibody or antibody fragment or antibody conjugate will vary depending on the specific activity of the antibody or fragment and/or the purpose for which the composition is to be used. Accordingly, this term is not to be construed to limit the invention to a specific quantity, e.g., weight or concentration, unless specifically stated otherwise. Methods for assessing efficacy of any amount of an antibody or antibody fragment or antibody conjugate for treating or preventing GAS infection or complication thereof in a subject or a diseases associated with GAS infection will be apparent to the skilled artisan from the disclosure herein. For example, a composition comprising an amount of an antibody or antibody fragment or antibody conjugate is administered to a population of subjects infected with GAS, and the number of subjects in which the severity of GAS infection or a symptom thereof or a complication thereof or a disease caused by GAS infection is reduced in determined. An amount of an antibody or antibody fragment or antibody conjugate that reduces the severity of GAS infection or a symptom thereof or a complication thereof or a disease caused by GAS infection in a significant proportion of the population is considered to be an amount of an antibody, antibody fragment or antibody conjugate sufficient to treat and/or prevent GAS infection or complication thereof in a subject or a disease or complication associated with GAS infection in a subject. For example, an effective amount of antibody, fragment or conjugate reduces the severity of GAS infection or a symptom thereof or a complication thereof or a disease caused by GAS infection in at least about 50% of the population or at least about 60% of the population or at least about 70% of the population or at least about 80% of the population or at least about 90% of the population or at least about 95% of the population or at least about 99% of the population.

The term “antibody” as used according to any embodiment hereof and unless the context requires otherwise shall be taken to mean any specific binding substance having a binding domain with the required specificity and/or affinity for an M protein B-cell epitope, including an immunoglobulin, antibody fragment e.g., V_{H}, V_{L}, Fab, Fab′, F(ab)₂, Fv, etc., having binding specificity and/or affinity for an M protein B-cell epitope, or an antibody conjugate comprising such antibodies and/or antibody fragments. The term “antibody” shall also be taken to include a cell expressing an antibody or antibody fragment or antibody conjugate, for example a hybridoma or plasmacytoma expressing a monoclonal antibody or a cell expressing a recombinant antibody fragment or a humanized antibody fragment or a chimeric antibody fragment. Preferred “antibodies” within this definition include intact polyclonal or monoclonal antibodies, an immunoglobulin (IgA, IgD, IgG, IgM, IgE) fraction, a chimeric antibody, a humanized antibody, an antibody fragment, or an immunoglobulin binding domain, whether natural or synthetic, and conjugates comprising same. Chimeric molecules including an immunoglobulin binding domain, or equivalent, fused to another polypeptide are also included within the meaning of the term “antibody” as used herein.

Preferred antibodies, antibody fragments and antibody conjugates are reactive with a conformational epitope of an M-protein of S. pyogenes (GAS) and only minimally reactive or non-reactive with a tissue of a subject to whom the antibody is administered.

Preferably, an antibody, antibody fragment or antibody conjugate is produced by a process comprising immunizing an animal with an immunogenic peptide composition comprising a chimeric peptide comprising a first amino acid sequence comprising a conformational epitope of an M-protein of S. pyogenes (GAS) linked to or within a second amino acid sequence having the same native conformation as the first sequence. Preferably, the immunogenic peptide composition further comprises a carrier protein e.g., diphtheria toxoid (DT) protein, preferably conjugated to the chimeric peptide.

A preferred conformational epitope of an M-protein of S. pyogenes (GAS) comprises the sequence REAK (SEQ ID NO: 14), and more preferably a sequence selected from the group consisting of:

| | | |
|---|---|---|
| (i) | LRRDLDASREAK; | (SEQ ID NO: 15) |
| (ii) | RRDLDASREAKK; | (SEQ ID NO: 16) |
| (iii) | RDLDASREAKKQ; | (SEQ ID NO: 17) |
| (iv) | DLDASREAKKQV; | (SEQ ID NO: 18) |
| (v) | LDASREAKKQVE; | (SEQ ID NO: 19) |
| (vi) | DASREAKKQVEK; | (SEQ ID NO: 20) |
| (vii) | ASREAKKQVEKA; | (SEQ ID NO: 21) |
| (viii) | SREAKKQVEKAL; | (SEQ ID NO: 22) |
| (ix) | REAKKQVEKALE; | (SEQ ID NO: 23) |
| (x) | ASREAKKQVEKALE; | (SEQ ID NO: 24) |

| | |
|---|---|
| and | |
| (xi) | mixtures of any one or more of (i) to (x). |

More preferably, a conformational epitope of an M-protein of S. pyogenes (GAS) comprises the sequence SREAKKQVEKAL (SEQ ID NO: 22) or ASREAKKQVEKALE (SEQ ID NO: 24) or a mixture thereof and still more preferably, the sequence SREAKKQVEKAL (SEQ ID NO: 22).

A preferred sequence in which a conformational epitope of an M protein of S. pyogenes (GAS) is embedded for the purpose of producing such antibodies, antibody fragments and antibody conjugates is a heptad repeat sequence such as, but not limited to, VKQ(L/A) ED(KQ) (SEQ ID NO: 25).

In one example, an antibody, antibody fragment or antibody conjugate is produced by a process comprising immunizing an animal with a chimeric peptide comprising a sequence selected from the group consisting of:

(i)

| | |
|---|---|
| QLEDKVKQLRRDLDASREAKEELQDKVK; | (SEQ ID NO: 2) |
| (ii) | |
| LEDKVKQARRDLDASREAKKELQDKVKQ; | (SEQ ID NO: 3) |
| (iii) | |
| EDKVKQAERDLDASREAKKQLQDKVKQL; | (SEQ ID NO: 4) |
| (iv) | |
| DKVQKAEDDLDASREAKKQVQDKVKQLE; | (SEQ ID NO: 5) |
| (v) | |
| KVKQAEDKLDASREAKKQVEDKVKQLED; | (SEQ ID NO: 6) |
| (vi) | |
| VKQAEDKVDASREAKKQVEKKVKQLEDK; | (SEQ ID NO: 7) |
| (vii) | |
| KQAEDKVKASREAKKQVEKAVKQLEDKV; | (SEQ ID NO: 8) |
| (viii) | |
| QAEDKVKQSREAKKQVEKALKQLEDKVQ; | (SEQ ID NO: 9) |
| (ix) | |
| AEDKVKQLREAKKQVEKALEQLEDKVQL; | (SEQ ID NO: 10) |
| (x) | |
| KQAEDKVKASREAKKQVEKALEQLEDKVK; | (SEQ ID NO: 11) |
| and | |
| (xi) | |

mixtures of any one or more of (i) to (x).

As used herein, the term “chimeric peptide immunogen” shall be taken to mean any peptide, or polypeptide of the M-protein of Group A streptococci comprising a conformational B-cell epitope wherein a therapeutic antibody, antibody fragment or antibody conjugate prepared using the chimeric peptide antigen is reactive with said B cell epitope and only minimally reactive with tissues of a subject to which the antibody, antibody fragment or antibody conjugate is administered.

The chimeric peptides used as immunogens are preferably derived from within a conserved region of peptide p145 and comprises one or more of the peptides J1 to J9, J14 and Jcon as shown in FIG. 1 (SEQ ID NOs: 1-11 and 26). Any other peptide that induces a protective immune response against GAS and disclosed in WO 96/11944 is incorporated by reference.

Preferred antibodies are immunoglobulin fractions or monoclonal antibodies or recombinant antibodies or humanized versions thereof.

By “humanized antibody” is meant an antibody, antibody fragment or antibody conjugate comprising variable region framework residues substantially from, for example, a human antibody (termed an acceptor antibody) and complementarity determining regions substantially from, for example, a mouse-antibody, (referred to as the donor immunoglobulin). Constant region(s), if present, is(are) substantially or entirely from a human immunoglobulin. The human variable domains are usually chosen from human antibodies whose framework sequences exhibit a high degree of sequence identity with a murine variable region domain from which the CDR/s were derived. The heavy and light chain variable region framework residues can be derived from the same or different human antibody sequences. The human antibody sequences can be the sequences of naturally-occurring human antibodies or can be consensus sequences of several human antibodies (e.g., as described in WO 92/22653).

The antibody, antibody fragment or antibody conjugate may be of any immunoglobulin isotype e.g., IgM, IgA, IgD, IgE, IgG, including e.g., IgG1, IgG2, etc.

In another example, an antibody conjugate is employed e.g., comprising an antibody or antibody fragment having the desired specificity for an epitope of the M-protein of GAS conjugated to a toxic agent. The invention clearly extends to any and all such antibody conjugates. Conjugates comprising toxins are particularly useful for targeted cytotoxicity of GAS cells. Suitable toxic substances for the production of toxin-containing antibody conjugates will be apparent to the skilled artisan and include, for example, paclitaxol, cytochalasin B, gramicidin D, ethidium bromide, emetine, mitomycin, puromycin and analogs or homologs thereof.

Preferably, the antibodies, antibody fragments and antibody conjugates are modified to enhance their stability in vivo. It will be appreciated by those skilled in the art that a variety of methods may be used to modify the therapeutic antibodies, fragments and conjugates such that their in vivo stability is increased thereby enhancing the effective serum titer of a unit dose. For example, the antibody, antibody fragment or antibody conjugate is PEGylated, and/or the sequence of the immunogenic moiety of a recombinant antibody or antibody fragment is modified to remove one or more protease cleavage sites.

As used herein, the term “treat” or variations thereof such as “treatment” shall be taken to mean a treatment following GAS infection that results in reduced bacterial count, prevention or reduction in severity of one or more symptoms of GAS infection e.g., uncomplicated pharyngitis, pyoderma, skin infection, soft tissue infection, bacteremia, necrotizing fasciitis, rheumatic fever, rheumatic heart disease, acute glomerulonephritis or morbidity, amongst others. It is to be understood that such treatment therefore includes the prophylaxis of GAS infection in so far as it prevents or reduces symptom development in an infected individual and/or prevents development of a complication thereof. Alternatively, or in addition, treatment in the present context also includes the prophylaxis of GAS infection in so far as it prevents or reduces an increase in bacterial load in an infected individual.

The composition comprising an antibody, antibody fragment or antibody conjugate that binds to an epitoe of an M-protein of GAS as described herein is particularly useful for treating GAS infection or complication thereof in a human or other mammalian subject or for treating a disease associated with GAS infection in a human or other mammalian subject. Preferably, the composition is for the treatment of humans.

Without compromising the generality of such compositions for the treatment of immunized and non-immunized subjects alike, the composition comprising an antibody, antibody fragment or antibody conjugate as described according to any embodiment hereof is particularly suited to the prophylactic and/or therapeutic treatment of non-vaccinated or immune-compromized or immune-deficient subjects. By “non-vaccinated” is meant that the subject has not been vaccinated with a peptide-based vaccine comprising an immunogenic peptide derived from a protein of Streptococcus pyogenes. By “immune-compromized” is meant that the subject does not produce endogenous antibody at a level sufficient to prevent the spread or development of GAS infection or the progression of disease arising from GAS infection, as a consequence of infection by another disease agent, radiation damage, treatment (e.g., chemotherapy) or general ill-health, e.g., a subject that is HIV+. By “immune-deficient” is meant that the subject does not have a functional immune system sufficient to produce endogenous antibody at a level to prevent the spread or development of GAS infection or the progression of disease arising from GAS infection, as a consequence of a genetic defect, radiation damage, treatment (e.g., chemotherapy). In immune-compromized and/or immune-deficient subjects, antibody against a B-cell epitope of GAS may not be produced at detectable levels, or at a level that reflects bacterial burden.

The present invention also provides a pharmaceutical composition comprising a unit dose of one or more antibodies, antibody fragments and antibody conjugates as described according to any embodiment hereof and a pharmaceutically acceptable carrier or excipient. Such compositions are formulated without undue experimentation for intravenous, intranasal, intramuscular, oral, subcutaneous, or intradermal delivery, or via suppository or implant (eg using slow release molecules). Preferred unit doses of antibody, antibody fragment or antibody conjugate generally comprise from about 0.1 μg immunoglobulin per kilogram body weight to about 100 mg immunoglobulin per kilogram body weight, preferably from about 0.1 μg immunoglobulin per kilogram body weight to about 20 mg immunoglobulin per kilogram body weight, more preferably from about 0.1 μg immunoglobulin per kilogram body weight to about 10 mg immunoglobulin per kilogram body weight, and still more preferably from about 0.1 μg immunoglobulin per kilogram body weight to about 1.0 mg immunoglobulin per kilogram body weight. Suitable carriers and excipients will vary according to the mode of administration and storage requirements of a composition comprising an antibody, antibody fragment or antibody conjugate and are described herein.

As used herein, the term “suitable carrier or excipient” shall be taken to mean a compound or mixture thereof that is suitable for use in a composition for administration to a subject for the treatment of GAS infection or complication thereof in a subject or a disease or complication associated with GAS infection in a subject. For example, a suitable carrier or excipient for use in a pharmaceutical composition for injection into a subject will generally not cause an adverse response in a subject.

A carrier or excipient useful in the pharmaceutical composition will generally not inhibit to any significant degree a relevant biological activity of an antibody, antibody fragment or antibody conjugate as described according to any embodiment hereof e.g., the carrier or excipient will not significantly inhibit the ability of an antibody, fragment or conjugate to bind to an M protein of GAS and/or to prevent the growth or spread of GAS cells and/or to kill GAS cells. For example, a carrier or excipient may merely provide a buffering activity to maintain the active compound at a suitable pH to thereby exert its biological activity, e.g., phosphate buffered saline. Alternatively, or in addition, the carrier or excipient may comprise a compound that enhances the activity or half-life of the antibody, antibody fragment or antibody conjugate e.g., a protease inhibitor. In yet another example, the carrier or excipient may include an antibiotic and/or an anti-inflammatory compound.

It will be appreciated by those skilled in the art that the invention also encompasses sustained release compositions comprising one or more antibodies, antibody fragments or antibody conjugates as described according to any embodiment hereof, e.g., to reduce the dosage required and/or frequency of administration to a subject and/or to prolong serum titer following administration.

The present invention provides a composition comprising an amount of a nucleic acid encoding an antibody or antibody fragment described according to any embodiment hereof that is sufficient to treat and/or prevent GAS infection or complication thereof in a subject or a disease or complication associated with GAS infection in a subject wherein said antibody or fragment binds immunospecifically to a B-cell epitope of Group A streptococci (GAS) M-protein.

Preferably, the nucleic acid is operably-linked to a promoter that induces expression of said antibody or antibody fragment in a cell, tissue or organ of a subject to whom it is administered. For example, in the case of a nucleic acid for administration to a human subject, the nucleic acid encoding the antibody or antibody fragment is operably linked to a promoter capable of inducing expression of said antibody or fragment in a human cell, tissue or organ. Suitable promoters will be apparent to the skilled artisan and include for example, an immediate early promoter from human cytomegalovirus or a SV40 promoter.

The present invention also provides an isolated cell expressing an antibody, antibody fragment or antibody conjugate as described according to any embodiment hereof e.g., example, a hybridoma or plasmacytoma expressing the antibody or antibody fragment or a cell expressing a recombinant antibody or recombinant antibody fragment, or a conjugate comprising such antibodies or antibody fragments.

The present invention also provides a composition comprising an amount of one or more cells expressing an antibody, antibody fragment or antibody conjugate sufficient to treat and/or prevent GAS infection or complication thereof in a subject or a disease or complication associated with GAS infection in a subject wherein said antibody, antibody fragment or antibody conjugate binds immunospecifically to a B-cell epitope of Group A streptococci (GAS) M-protein.

Preferably, the cell is a cell expressing a recombinant antibody or a fragment thereof or a conjugate comprising said antibody or fragment. For example, the cell is a cell from a subject to be treated, e.g., a blood cell from a subject, e.g., a leukocyte cell from a subject.

The present invention also provides a method for producing a composition described herein according to any embodiment. For example, in its broadest form, such a method comprises mixing or otherwise combining: (i) an amount of an antibody, antibody fragment or antibody conjugate or nucleic acid encoding said antibody, antibody fragment or antibody conjugate or a cell expressing said antibody, antibody fragment or antibody conjugate sufficient to treat and/or prevent GAS infection or complication thereof in a subject or a disease or complication associated with GAS infection in a subject, wherein said antibody, antibody fragment or antibody conjugate binds immunospecifically to a B-cell epitope of Group A streptococci (GAS) M-protein; and (ii) a suitable carrier or excipient.

In one example, the method additionally comprises producing or obtaining an integere selected from the antibody, antibody fragment, antibody conjugate, nucleic acid encoding said antibody, nucleic acid encoding said antibody fragment, nucleic acid encoding said antibody conjugate, a cell expressing said antibody, a cell expressing said antibody fragment, a cell expressing said antibody conjugate and mixtures thereof. For example, an antibody or antibody fragment is produced recombinantly or isolated from a cell expressing same, using a method known in the art and/or described herein.

The present invention also provides a method of treating or ameliorating GAS infection in a human or other mammalian subject said method comprising administering to said subject a composition as described according to any embodiment hereof, wherein said composition is administered in an amount effective to prevent an increase in bacterial count or to reduce bacterial count in a sample from the subject.

In an alternative embodiment, the present invention also provides a method of preventing, ameliorating or treating a disease or complication associated with GAS infection of a human or other mammalian subject said method comprising administering to said subject a composition as described according to any embodiment hereof, wherein said composition is administered in an amount effective to reduce the severity of one or more disease symptoms or to prevent onset of one or more diseases arising from GAS infection.

In a further alternative embodiment, the present invention also provides a method of neutralizing a GAS pathogen in a subject exposed to the pathogen said method comprising administering to a subject infected with GAS pathogen a composition as described according to any embodiment hereof, wherein said composition is administered in an amount effective to opsonize said pathogen in the serum of the subject. The term “opsonize” as used herein should be construed as promoting the ingestion and killing of GAS by phagocytic cells in the subject.

Preferably, the composition is administered for a time and under conditions sufficient to achieve the stated purpose, e.g., to prevent an increase in bacterial count or to reduce bacterial count in a sample from the subject or to reduce the severity of one or more disease symptoms or to prevent onset of one or more diseases arising from GAS infection or to opsonize a GAS pathogen in the serum of the subject. For example, the composition is administered by continuous infusion, or is administered a plurality of times to thereby achieve the stated purpose.

Preferred diseases or complications associated with GAS infection in the present context include, but are not limited to, uncomplicated pharyngitis, pyoderma, skin infection, soft tissue infection, bacteremia, necrotizing fasciitis, rheumatic fever, rheumatic heart disease, acute glomerulonephritis, or morbidity, amongst others.

Preferably, the pharmaceutical composition is co-administered with an antibiotic having bacteriostatic or bacteriocidal activity against S. pyogenes (GAS). By “co-administered” is meant that the antibiotic is administered to the subject for the purposes of treating the same infection as the pharmaceutical composition of the invention, irrespective of whether or not antibiotic and pharmaceutical composition are administered in the same or a different unit dose or at the same or a different time. Generally, albeit not necessarily, the antibiotic and pharmaceutical composition will be administered in different unit doses. Such compounds are administered by well-established routes, generally orally or by intravenous or intramuscular injection. In a particularly preferred embodiment, the antibiotic is a penicillin compound e.g., amoxicillin, erythromycin, cephalexin, cefadroxil, cefaclor, cefuroxime axatil, cefizime, cefdinir, penicillin VK, penicillin G benzathine, or a mixture thereof. The co-administration of other antibiotics is not to be excluded.

The subject is preferably a non-vaccinated or immune-compromized or immune-deficient subject. Alternatively, or in addition, the subject is an individual that has been vaccinated against a strain of S. pyogenes wherein said vaccination has not resulted in protection sufficient to prevent a subsequent infection or to prevent the onset of disease associated with infection thereby. Alternatively, or in addition, the subject is an individual that has been vaccinated against a strain of S. pyogenes however is immune-compromized or immune-deficient.

Preferably, the subject is human.

As used herein, the term “amount effective” refers to the amount of a therapeutic composition comprising an antibody, antibody fragment or antibody conjugate that binds to an epitoe of an M-protein of GAS as described herein, is sufficient to reduce the severity, and/or duration of a GAS infection; ameliorate one or more symptoms thereof, prevent the advancement of a GAS infection, or cause regression of a GAS infection, or which is sufficient to result in the prevention of the development, recurrence, onset, or progression of a GAS infection or one or more symptoms thereof, or enhance or improve the prophylactic and/or therapeutic effect(s) of another therapy (e.g., another therapeutic agent).

The efficacy of treatment is established by any means known to the skilled artisan e.g., by determining live cell count in a sample from the subject such as, for example, serology based on cultures from clinical specimens such as sera or throat swab. For example, serologic methods can detect group A antigen; or by the precipitin test. Alternatively, or in addition the efficacy of treatment is determined by determining bacitracin sensitivity of a clinical specimen. Bacitracin sensitivity presumptively differentiates group A from other b-hemolytic streptococci (B, C, G). Alternatively, or in addition, acute glomerulonephritis and acute rheumatic fever are identified by anti-streptococcal antibody titres in serum from a subject. In addition, diseases associated with GAS infection such as acute rheumatic fever are diagnosed by clinical criteria.

In use, the antibody, antibody fragment or antibody conjugate is preferably bacteriostatic or bacteriocidal. Preferably, the antibody, antibody fragment or antibody conjugate is bacteriocidal. Such bactericidal activity can be conferred by a toxic agent conjugated to an antibody, antibody fragment or antibody conjugate. Alternatively or In addition, the antibody, antibody fragment or antibody conjugate can have bactericidal activity without the need for a toxin conjugate, e.g., by inducing antibody dependent cell cytotoxicity.

The present invention also provides a method of maintaining a therapeutically or prophylactically effective serum titer of an antibody against an M protein of S. pyogenes (GAS) in a subject said method comprising administering a plurality of doses of a composition as described according to any embodiment hereof, wherein said each of said doses is administered in an amount effective to prevent an increase in bacterial count or to reduce bacterial count in a sample from the subject and/or to reduce the severity of one or more disease symptoms or to prevent onset of one or more diseases arising from GAS infection and/or to opsonize said pathogen in the serum of the subject. Preferably, the method further comprises monitoring antibody titre in the serum of the subject to thereby determine when antibody titres are declining in the serum. Preferably, second and subsequent doses of the composition are administered when antibody titres in serum are decreasing e.g., after antibody titres commence their decline and/or before they have reached a minimum level in the serum.

In one example, a method as described according to any embodiment hereof additionally comprises providing or obtaining a composition as described according to any embodiment hereof or information concerning same. For example, the present invention provides a method of treating or ameliorating GAS infection in a human or other mammalian subject or preventing, ameliorating or treating a disease or complication associated with GAS infection of a human or other mammalian subject or neutralizing a GAS pathogen in a subject exposed to the pathogen, said method comprising:

(i) determining a subject suffering from a GAS infection or a disease or complication associated with GAS infection or at risk of developing a GAS infection or a disease or complication associated with GAS infection;
(ii) obtaining a composition as described according to any embodiment hereof; and
(iii) administering said composition to said subject.

In another example, a method of treating or ameliorating GAS infection in a human or other mammalian subject or preventing, ameliorating or treating a disease or complication associated with GAS infection of a human or other mammalian subject or neutralizing a GAS pathogen in a subject exposed to the pathogen, said method comprising:

(i) identifying a subject suffering from a GAS infection or a disease or complication associated with GAS infection or at risk of developing a GAS infection or a disease or complication associated with GAS infection; and
(ii) recommending administration of a composition as described according to any embodiment hereof.

Alternatively, the present invention provides a method of treating or ameliorating GAS infection in a human or other mammalian subject or preventing, ameliorating or treating a disease or complication associated with GAS infection of a human or other mammalian subject or neutralizing a GAS pathogen in a subject exposed to the pathogen said method comprising administering or recommending administration of a composition as described according to any embodiment hereof to a subject previously identified as suffering from a GAS infection or a disease or complication associated with GAS infection or at risk of developing a GAS infection or a disease or complication associated with GAS infection.

The present invention also provides an amount of an antibody, antibody fragment or antibody conjugate that binds immunospecifically to a B-cell epitope of Group A streptococci (GAS) M-protein, or nucleic acid encoding said antibody, antibody fragment or antibody conjugate, or a cell expressing said antibody, antibody fragment or antibody conjugate, or a composition comprising said antibody, antibody fragment, antibody conjugate, nucleic acid, or cell, for use in medicine e.g., for therapy of an acute severe GAS infection or a disease or complication associated therewith, such as uncomplicated pharyngitis, pyoderma, skin infection, soft tissue infection, bacteremia, necrotizing fasciitis, rheumatic fever, rheumatic heart disease, acute glomerulonephritis, or morbidity.

The present invention also provides an amount of an antibody, antibody fragment or antibody conjugate that binds immunospecifically to a B-cell epitope of Group A streptococci (GAS) M-protein, or nucleic acid encoding said antibody, antibody fragment or antibody conjugate, or a cell expressing said antibody, antibody fragment or antibody conjugate, in the preparation or manufacture of a medicament for the treatment of an acute severe GAS infection or a disease or complication associated therewith, such as uncomplicated pharyngitis, pyoderma, skin infection, soft tissue infection, bacteremia, necrotizing fasciitis, rheumatic fever, rheumatic heart disease, acute glomerulonephritis, or morbidity.

The present invention also provides a method for isolating an antibody, antibody fragment or antibody conjugate for treating or ameliorating GAS infection in a human or other mammalian subject or for preventing, ameliorating or treating a disease or complication associated with GAS infection of a human or other mammalian subject or for neutralizing a GAS pathogen in a subject exposed to the pathogen said method comprising:

(i) producing, isolating or obtaining an antibody, antibody fragment or antibody conjugate that binds immunospecifically to a B-cell epitope of GAS M-protein;
(ii) determining the ability of the antibody, antibody fragment or antibody conjugate to reduce or prevent growth of GAS or to reduce the severity, and/or duration of a GAS infection or ameliorate one or more symptoms thereof or prevent the advancement of a GAS infection, or cause regression of a GAS Infection; and
(iii) isolating an antibody, antibody fragment or antibody conjugate from (ii) that reduces or prevents growth of GAS or reduces the severity, and/or duration of a GAS infection or ameliorates one or more symptoms thereof or prevents the advancement of a GAS infection, or causes regression of a GAS infection, thereby isolating an antibody for treating or ameliorating GAS infection in a human or other mammalian subject or for preventing, ameliorating or treating a disease or complication associated with GAS infection of a human or other mammalian subject or for neutralizing a GAS pathogen in a subject exposed to the pathogen.

In one example, the method further comprises establishing a correlation between affinity of an antibody, antibody fragment or antibody conjugate for an epitope of an M-protein of GAS and a desired bioactivity e.g., activity against GAS cell viability or growth or cell division. By virtue of establishing such a correlation, it is also possible to then perform a modification of this method by:

(i) producing, isolating or obtaining an antibody, antibody fragment or antibody conjugate that binds to a B-cell epitope of an M-protein of GAS; and
(ii) isolating an antibody, antibody fragment or antibody conjugate from (i) that binds at a desired affinity to a B-cell epitope of an M-protein of GAS, thereby isolating an antibody for treating or ameliorating GAS infection in a human or other mammalian subject or for preventing, ameliorating or treating a disease or complication associated with GAS infection of a human or other mammalian subject or for neutralizing a GAS pathogen in a subject exposed to the pathogen.

In one example, the antibody, antibody fragment or antibody conjugate binds immunospecifically to a B-cell epitope comprising a sequence set forth in any one or more of SEQ ID NOs: 2-12 or 15-24 or 26. Preferably, the antibody, antibody fragment or antibody conjugate binds immunospecifically to a B-cell epitope comprising the sequence set forth in SEQ ID NO: 9.

Methods for producing or isolating antibodies, fragments and conjugates will be apparent to the skilled artisan and/or described herein. In one example, an immunogen is administered to a subject to thereby produce a monoclonal antibody, polyclonal antisera, and optionally an antibody fragment and/or antibody conjugate is produced from the monoclonal or polyclonal antibody. Alternatively, nucleic acid encoding a recombinant antibody or antibody fragment is mutated e.g., by random mutagenesis or targeted mutation and recombinant antibodies or antibody fragments encoded by the nucleic acid are expressed using a suitable expression system. Alternatively or in addition, an antibody or antibody fragment is subjected to affinity maturation to produce a high(er) affinity molecule than the immature parent molecule.

In one example, the method additionally comprises determining affinities of a plurality of antibodies, antibody fragments or antibody conjugates for to a B-cell epitope of an M-protein of GAS and/or determining abilities of a plurality of antibodies, antibody fragments or antibody conjugates, or a library of antibodies, antibody fragments or antibody conjugates to reduce or prevent growth of GAS or to reduce the severity, and/or duration of a GAS infection or ameliorate one or more symptoms thereof or prevent the advancement of a GAS infection, or cause regression of a GAS infection.

Preferably, an antibody, antibody fragment or antibody conjugate or a mixture thereof that binds at a desired affinity to a B-cell epitope of an M-protein of GAS, and/or reduces or prevents growth of GAS or reduces the severity, and/or duration of a GAS infection or ameliorates one or more symptoms thereof or prevents the advancement of a GAS infection, or causes regression of a GAS infection, is then separated and/or isolated and/or purified from said plurality. In accordance with this embodiment, the term “separating” can comprise the use of any chemical or biochemical purification process known in the art to fractionate the mixture of plurality of antibodies, antibody fragments or antibody conjugates coupled with assaying the fractions produced for their affinities for a B-cell epitope of an M-protein of GAS and/or their bioactivities e.g., an ability reduce or prevent growth of GAS or to reduce the severity, and/or duration of a GAS infection or ameliorate one or more symptoms thereof or prevent the advancement of a GAS infection, or cause regression of a GAS infection. The term “separating” can also refer to a process comprising iterations of any chemical or biochemical purification process known in the art to partially or completely purify an antibody, antibody fragments or antibody conjugates from a mixture or plurality of antibodies, antibody fragments or antibody conjugates, and assaying the fractions produced in each iteration of the process for their affinities for a B-cell epitope of an M-protein of GAS and/or their bioactivities e.g., their ability to reduce or prevent growth of GAS or to reduce the severity, and/or duration of a GAS infection or ameliorate one or more symptoms thereof or prevent the advancement of a GAS infection, or cause regression of a GAS infection. Preferably, n iterations are performed, wherein n is sufficient number of iterations to reach a desired purity of the antibody, antibody fragment or antibody conjugate e.g., 50% or 60% or 70% or 80% or 90% or 95% or 99%. More preferably, the process is repeated for zero to about ten iterations. As will be known to the skilled artisan, such iterations do not require iteration of precisely the same purification processes and more generally utilize different processes or purification conditions for each iteration. In the case of starting material comprising a plurality of distinct antibodies, fragments and conjugates e.g., displayed separately wherein each antibody, antibody fragment or antibody conjugate is substantially pure prior to performance of the method, such isolation results in the separation of the antibody, fragment or conjugate from other antibodies, fragments and conjugates that do not have the requisite bioactivity or affinity for an epitope of an M-protein of GAS. In this case, the term “separating” extends to determining the activity of one antibody, fragment or conjugate relative to another antibody, fragment or conjugate in the mix, and selecting a antibody, fragment or conjugate having the desired activity or affinity for an epitope of an M-protein of GAS.

In a further example, the method additionally comprises determining the amino acid sequence of an isolated antibody or antibody fragment or the immunogenic moiety of an antibody conjugate having a desired affinity for a B-cell epitope of an M-protein of GAS and/or a desired bioactivity e.g., an antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection.

In a further example, the method according to any embodiment supra additionally comprises:
(a) optionally, determining the amino acid sequence of an isolated antibody, antibody fragment or moiety of an antibody conjugate having a desired affinity for an epitope of an M-protein of GAS and/or a desired bioactivity e.g., an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection;
(b) optionally, providing the name or sequence of an isolated antibody, antibody fragment or moiety of an antibody conjugate having a desired affinity for an epitope of an M-protein of GAS and/or a desired bioactivity e.g., an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection; and
(c) providing the isolated antibody, antibody fragment or moiety of an isolated antibody conjugate having a desired affinity for an epitope of an M-protein of GAS and/or a desired bioactivity e.g., an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection.

In a further example, the method additionally comprises determining the sequence of nucleic acid encoding an isolated antibody or antibody fragment or the immunogenic moiety of an antibody conjugate having a desired affinity for a B-cell epitope of an M-protein of GAS and/or a desired bioactivity e.g., the sequence of nucleic acid encoding an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection.

In a further example, the method according to any embodiment supra additionally comprises:
(a) optionally, determining the sequence of nucleic acid encoding an isolated antibody, antibody fragment or moiety of an antibody conjugate having a desired affinity for an epitope of an M-protein of GAS and/or a desired bioactivity e.g., the sequence of nucleic acid encoding an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection;
(b) optionally, providing the sequence of nucleic acid encoding an isolated antibody, antibody fragment or moiety of an antibody conjugate having a desired affinity for an epitope of an M-protein of GAS and/or a desired bioactivity e.g., the sequence of nucleic acid encoding an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection; and
(c) providing the sequence of nucleic acid encoding an isolated antibody, antibody fragment or moiety of an isolated antibody conjugate having a desired affinity for an epitope of an M-protein of GAS and/or a desired bioactivity e.g., the sequence of nucleic acid encoding an isolated antibody, fragment or conjugate that reduces or prevents growth of GAS or reduces the severity, and/or duration of a GAS infection or ameliorates one or more symptoms thereof or prevents the advancement of a GAS infection, or causes regression of a GAS infection.

In a further example, the method additionally comprises:
(iv) optionally, determining the amino acid sequence of an antibody, antibody fragment or moiety of an antibody conjugate having a desired affinity for an epitope of an M-protein of GAS, or a desired biological activity e.g., activity against GAS cell viability or growth or cell division;
(v) optionally, providing the name or sequence of the antibody, antibody fragment or moiety of an antibody conjugate at (iv); and
(viii) providing the antibody, antibody fragment or moiety of an antibody conjugate at (iv).

In one example, the method additionally comprises providing or obtaining a plurality of antibodies, antibody fragments or antibody conjugates or a library of antibodies, antibody fragments or antibody conjugates.

The present invention clearly extends to the direct product of any method of isolation of an antibody as described according to any embodiment hereof.

It is to be understood that an isolated antibody, antibody fragment or antibody conjugate in substantially pure form i.e., free from contaminants that might cause adverse side effects or contraindications or antagonize the affinity or bioactivity of the antibody, fragment or conjugate, can be formulated into a composition for use in a method as described according to any embodiment hereof. Accordingly, in one example, the present invention additionally comprises formulating the isolated antibody, fragment or conjugate with a suitable carrier or excipient.

### DEFINITIONS

Unless the context requires otherwise or specifically stated to the contrary, integers, steps, or elements of the invention recited herein as singular integers, steps or elements clearly encompass both singular and plural forms of the recited integers, steps or elements.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

The invention described herein with respect to any embodiment in so far as it refers to treatment of an infection caused by GAS or complication thereof in a subject or a disease or complication associated with GAS infection in a subject shall be taken to apply mutatis mutandis to therapeutic treatment of an infection caused by GAS or complication thereof in a subject or a disease or complication associated with GAS infection in a subject.

The invention described herein with respect to any embodiment in so far as it refers to treatment of an infection caused by GAS or complication thereof in a subject or a disease or complication associated with GAS infection in a subject shall be taken to apply mutatis mutandis to prophylactic treatment of an infection caused by GAS or complication thereof in a subject or a disease or complication associated with GAS infection in a subject.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features. The present invention is also not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1 is a schematic representation of the sequence of chimeric peptides derived from p145. M-protein sequence is in bold and GCN4 derived sequence is not bold.

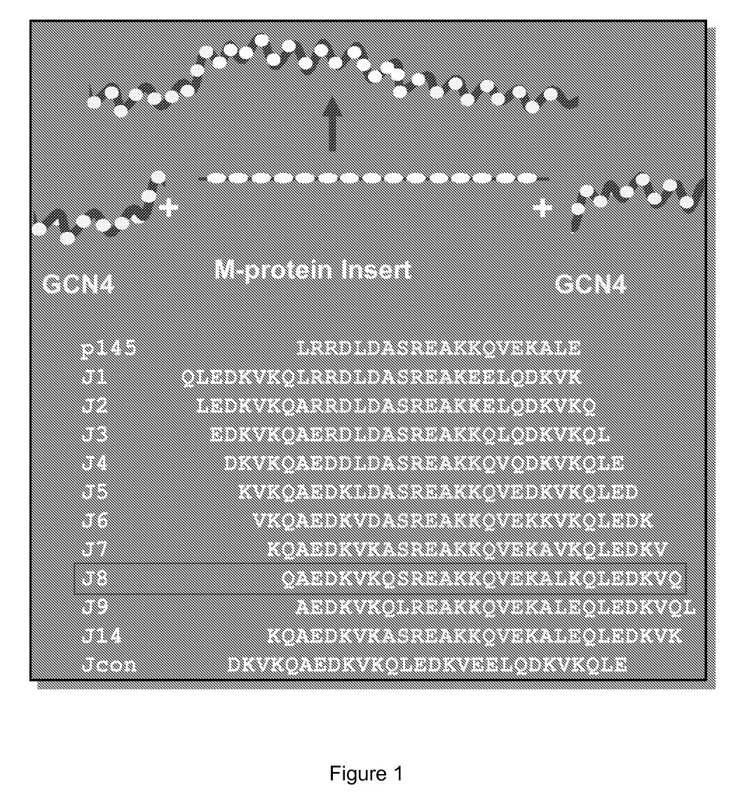

FIG. 2 is a graphical representation of the protective potential of passively transferred J8-DT antisera to BALB/c mice. Antisera were transferred into naïve animals, which were subsequently challenged with GAS M1 strain. The results were compared using Mann-Whitney test. Animals receiving J8-DT antiserum survived significantly better than animals receiving control PBS antiserum (p<0.05).

FIG. 3 is a graphical representation of the concentration of a J8-specific serum IgG in donor mice. Cohorts of BALB/c mice were immunised subcutaneously with J8-DT, DT or PBS alone in alum. Serum samples collected on day 42 were quantitated by ELISA. Antibody concentrations to J8 in serum of individual mice are shown, with average concentrations (geometric means) represented as bars.

FIG. 4 is a graphical representation of the concentration of J8-specific IgG in recipient BALB/c and SCID mice. Hyperimmune serum was transferred intraperitoneally into naïve BALB/c and SCID mice in three doses of 0.5 mL each. Serum samples were collected after second administration of antiserum. Antibody concentrations to J8 in serum of individual mice are shown, with average concentrations (geometric means) represented as bars.

FIG. 5 is a graphical representation of the protective potential of passively transferred J8-DT antisera to BALB/c and SCID mice. The curve shows survival of mice receiving parenteral prime-boost immunisation with J8-DT, DT and PBS or IP administration of J8-DT antisera followed by intraperitoneal challenge with GAS M1 strain. The abbreviation I/Ch stands for immunised/challenged BALB/c mice whereas other groups of mice are recipients of passively transferred serum. Mann-Whitney test was used to compare the proportions of surviving mice and significance is represented as * where p*<0.05, p**<0.01, p***0.001.

FIG. 6 is a graphical representation of the concentration of J8-specific serum IgG in recipient mice post challenge.

FIG. 7 is a graphical representation of the immunogenicity of J8-DT/alum in rabbits.

FIG. 8 is a graphical representation of J8-specific serum IGG in cohorts of recipient BALB/c or SCID mice which received either J8-DT, DT or control rabbit IgG.

FIG. 9 is a graphical representation of survival of recipient mice following intraperitoneal challenge with M1 GAS.

FIG. 10 is a graphical representation of mouse and rabbit J8-Specific serum IgG in the recipient mice following challenge.

FIG. 11 is graphical representation of antibody levels of BALB/c mice immunised and depleted/undepleted of CD4+ T-cells following an intraperitoneal M1 GAS challenge.

FIG. 12 is a graphical representation of percentage survival of BALB/c mice immunised and depleted/undepleted of CD4+ T-cells following an intraperitoneal M1 GAS challenge.

FIG. 13 is a graphical representation showing therapeutic treatment of BALB/c mice challenged with M1 GAS. Time following challenge (days) is indicated on the X-axis and percentage survival is shown on the Y-axis. J8-DT (I/Ch) indicates the percentage survival of mice immunized with the J8-DT peptide prior to challenge. J8-DT (PT/Ch) indicates percentage survival of naïve mice treated with anti-J8-DT antisera before and after challenge. J8-DT (T) indicates percentage survival of mice treated with anti-J8-DT antisera after challenge. DT(T) indicates percentage survival of mice treated with diphtheria toxoid antisera after challenge. PBS(I/Ch) indicates percentage survival of mice previously administered phosphate buffered saline (PBS) prior to challenge. PBS(T) indicates percentage survival of mice treated with PBS after challenge. Arrows indicate timing of administration of antiserum.

FIG. 14 is a graphical representation showing therapeutic treatment of SCID mice challenged with M1 GAS. Time following challenge (days) is indicated on the X-axis and percentage survival is shown on the Y-axis. J8-DT indicates the percentage survival of naïve mice treated with anti-J8-DT antisera after challenge. DT indicates percentage survival of mice treated with diphtheria toxoid after challenge. PBS indicates percentage survival of mice treated with PBS after challenge. Arrows indicate timing of administration of antiserum.

### DETAILED DESCRIPTION OF PREFERRED EMBODIMENTS

### Polyclonal Antibodies

In one example of the invention, a composition described herein according to any embodiment comprises a polyclonal antibody or polyclonal antisera. A polyclonal antibody is produced by immunizing a suitable animal, e.g., a rabbit, a mouse, a goat, or a rat, with an immunogen, e.g., a peptide as described herein in any embodiment, e.g., a peptide comprising a sequence set forth in any one or more of SEQ ID NOs: 2-12 or 15-24 or 26. Optionally, the immunogen is joined to a carrier protein, such as diptheria toxoid. The immunogen and optionally carrier protein is injected into the animal, preferably according to a predetermined schedule incorporating one or more booster immunizations, and blood collected from said animal periodically. Optionally, the immunogen is injected in the presence of an adjuvant, such as, for example Freund's complete or incomplete adjuvant, lysolecithin and/or dinitrophenol to enhance the immune response to the immunogen. Polyclonal antibodies specific for the GAS M1 antigen are then purified from the blood isolated from an animal by, for example, affinity chromatography using the GAS M1 antigen or the epitope used to immunize the animal, preferably coupled to a suitable solid support.

### Monoclonal Antibodies

In another example, the therapeutic antibody is a monoclonal antibody. A monoclonal antibody is produced by immunizing an animal (e.g., a mouse) with an immunogen, e.g., a peptide as described herein in any embodiment, e.g., a peptide comprising a sequence set forth in any one or more of SEQ ID NOs: 2-12 or 15-24 or 26. Optionally, the immunogen is injected in the presence of an adjuvant, such as, for example Freund's complete or incomplete adjuvant, lysolecithin and/or dinitrophenol to enhance the immune response to the immunogen. The immunogen may also be linked to a carrier protein, such as, for example, BSA. Spleen cells are then obtained from the immunized animal. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Alternatively, ABL-MYC technology (NeoClone, Madison Wis. 53713, USA) is used to produce cell lines secreting monoclonal antibodies (mAbs) immunoreactive with a B-cell epitope from GAS M protein. In this process, BALB/cByJ female mice are immunized with an amount of the peptide antigen over a period of about 2 to about 3 months. During this time, test bleeds are taken from the immunized mice at regular intervals to assess antibody responses in a standard ELISA. The spleens of mice having antibody titers of at least about 1,000 are used for subsequent ABL-MYC infection employing replication-incompetent retrovirus comprising the oncogenes v-abl and c-myc. Splenocytes are transplanted into naive mice which then develop ascites fluid containing cell lines producing monoclonal antibodies (mAbs) against the immunogen. The mAbs are purified from ascites using protein G or protein A, e.g., bound to a solid matrix, depending on the isotype of the mAb. Because there is no hybridoma fusion, an advantage of the ABL-MYC process is that it is faster, more cost effective, and higher yielding than conventional mAb production methods. In addition, the diploid palsmacytomas produced by this method are intrinsically more stable than polyploid hybridomas, because the ABL-MYC retrovirus infects only cells in the spleen that have been stimulated by the immunizing antigen. ABL-MYC then transforms those activated B-cells into immortal, mAb-producing plasma cells called plasmacytomas. A “plasmacytoma” is an immortalized plasma cell that is capable of uncontrolled cell division. Since a plasmacytoma begins with just one cell, all of the plasmacytomas produced from it are therefore identical, and moreover, produce the same desired “monoclonal” antibody. As a result, no sorting of undesirable cell lines is required. The ABL-MYC technology is described generically in detail in the following disclosures which are incorporated by reference herein:
1. Largaespada et al., Curr. Top. Microbiol. Immunol., 166, 91-96. 1990;
2. Weissinger et al., Proc. Natl. Acad. Sci. USA, 88, 8735-8739, 1991;
3. Largaespada et al., Oncogene, 7, 811-819, 1992;
4. Weissinger et al., J. Immunol. Methods 168, 123-130, 1994;
5. Largaespada et al., J. Immunol. Methods. 197 (1-2), 85-95, 1996; and
6. Kumar et al., Immuno. Letters 65, 153-159, 1999.

Monoclonal antibodies are isolated from the supernatants of growing hybridoma colonies or plasmacytomas using methods such as, for example, affinity purification using the immunogen used to immunize the animal to isolate an antibody capable of binding thereto. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

### Recombinant Antibodies

Methods for making recombinant antibodies are known in the art. For example, the sequence of a nucleic acid encoding a variable region of an antibody that immunospecifically binds to a B-cell epitope of a GAS M protein is isolated and placed in operable connection with a promoter that expresses a nucleic acid in a cell. For example, nucleic acid comprising a sequence that encodes an antibody as described according to any embodiment hereof in operable connection with a suitable promoter is introduced into a cell and the cell maintained for a time and under conditions sufficient for expression to occur.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid (e.g., a transgene) to which it is operably linked. Preferred promoters can contain additional copies of one or more specific regulatory elements to further enhance expression and/or alter the spatial expression and/or temporal expression of said nucleic acid.

As used herein, the term “in operable connection with” “in connection with” or “operably linked to” means positioning a promoter relative to a nucleic acid (e.g., a transgene) such that expression of the nucleic acid is controlled by the promoter. For example, a promoter is generally positioned 5′ (upstream) to the nucleic acid, the expression of which it controls. To construct heterologous promoter/nucleic acid combinations (e.g., promoter/transgene combinations), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

For expression in vitro of an antibody, antibody fragment or antibody conjugate that binds to an epitope of an M-protein of GAS or neutralizes GAS as described according to any embodiment hereof, it is preferred to utilize a T3 or a T7 bacteriophage promoter (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g., 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRatkneo (Muller et al., Mol. Cell. Biol., 11, 1785, 1991).

A wide range of additional host/vector systems suitable for expressing an antibody, antibody fragment or antibody conjugate are available publicly, and described, for example, in Sambrook et al (In: Molecular cloning, A laboratory manual, second edition, Cold Spring Harbor Laboratory, Cold Spring Harbor, N.Y., 1989).

Means for introducing an isolated nucleic acid or a gene construct comprising same or expression vector into a cell for expression are known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

Alternatively, a recombinant antibody is isolated from a phage display library. In phage display methods, functional antibody domains are displayed on the surface of phage particles which carry the polynucleotide sequences encoding them. In particular, DNA sequences encoding V_{H} and V_{L} domains are amplified from an animal cDNA library (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a B-cell epitope of a GAS M protein is selected or identified with antigen, e.g., using labeled antigen or antigen bound or captured to a solid surface or bead. Examples of phage display methods that are suitable for expressing an antibody or antibody fragment include those disclosed in Brinkman et al., 1995, J. Immunol. Methods 182:41-50; and U.S. Pat. Nos. 5,698,426, 5,223,409 and 5,403,484.

### Humanized Antibodies

In a preferred embodiment, the therapeutic antibody, antibody fragment or immunogenic moiety of an antibody conjugate is humanized, e.g., an antibody produced by molecular modelling techniques wherein the human content of the antibody is maximised while causing little or no loss of binding affinity attributable to the variable region of an antibody produced by a non-human antibody, e.g., a murine antibody. Thus, in one embodiment the invention provides a chimeric antibody comprising the amino acid sequence of a human framework region and of a constant region from a human antibody so as to humanise or render nonimmunogenic the hypervariable region from a mouse monoclonal antibody.

The methods described below are applicable to the humanization of a wide variety of animal antibodies. A two-step approach may be used which involves (a) selecting human antibody sequences that are used as human frameworks for humanization, and (b) determining which variable region residues of the animal monoclonal antibody should be selected for insertion into the human framework chosen.

The first step involves selection of the best available human framework sequences for which sequence information is available. This selection process is based upon the following selection criteria.

### (1) Percent Identities

The sequences of the heavy and light chain variable regions of an animal monoclonal antibody that is to be humanized are optimally aligned and compared preferably with a variety of human antibody heavy and light chain variable region sequences.

Once the sequences are thus compared, residue identities are noted and percent identities are determined. All other factors being equal, it is desirable to select a human antibody which has the highest percent identity with the animal antibody.

### (2) Sequence Ambiguitites

The known human antibody chain sequences are then evaluated for the presence of unidentified residues and/or ambiguities, which are sequence uncertainties. The most common of such uncertainties are mistaken identification of an acidic amino acid for an amide amino acid due to loss of ammonia during the sequencing procedure, e.g., incorrect identification of a glutamic acid residue, when the residue actually present in the protein was a glutamine residue. All other factors being equal, it is desirable to select a human antibody chain having as few such ambiguities as possible

### (3) Pin-Region Spacing

Antibody chain variable regions contain intra-domain disulfide bridges. The distance (number of residues) between the cysteine residues comprising these bridges is referred to as the Pin-region spacing [Chothia et al, J. Mol. Biol. 196:901 (1987)]. All other factors being equal, it is most desirable that the Pin-region spacing of a human antibody selected be similar or identical to that of the animal antibody. It is also desirable that the human sequence Pin-region spacing be similar to that of a known antibody 3-dimensional structure, to facilitate computer modeling.

Based upon the foregoing criteria, a human antibody (or antibodies) having the best overall combination of desirable characteristics is selected as the framework for humanization of the animal antibody. The heavy and light chains selected may be from the same or different human antibodies.

The second step in the method involves determination of which of the animal antibody variable region sequences should be selected for grafting into the human framework. This selection process is based upon the following selection criteria:

### (1) Residue Selection

Two types of potential variable region residues are evaluated in the animal antibody sequences, the first of which are called “minimal residues.” These minimal residues comprise CDR structural loops plus any additional residues required, as shown by computer modeling, to support and/or orient the CDR structural loops.

The other type of potential variable region residues are referred to as “maximal residues.” They comprise the minimal residues plus any additional residues which, as determined by computer modeling, fall within about 10/of CDR structural loop residues and possess a water solvent accessible surface [Lee et al, J. Biol. Chem. 55:379 (1971)].

### (2) Computer Modeling

To identify potential variable region residues, computer modeling is carried out on (a) the variable region sequences of the animal antibody that is to be humanized, (b) the selected human antibody framework sequences, and (c) all possible recombinant antibodies comprising the human antibody framework sequences into which the various minimal and maximal animal antibody residues have been grafted.

The computer modeling is performed using software suitable for protein modeling and structural information obtained from an antibody that (a) has variable region amino acid sequences most nearly identical to those of the animal antibody and (b) has a known 3-dimensional structure. An example of software that can be used is the SYBYL Biopolymer Module software (Tripos Associates). The antibody from which the structural information can be obtained may be but need not necessarily be a human antibody.

Based upon results obtained in the foregoing analysis, recombinant chains containing the animal variable regions producing a computer modeling structure most nearly approximating that of the animal antibody are selected for humanisation.

Human antibodies can also be produced using transgenic mice that are incapable of expressing functional endogenous immunoglobulins, however express human immunoglobulins. For example, the human heavy and light chain immunoglobulin gene complexes are introduced randomly or by homologous recombination into mouse embryonic stem cells. Alternatively, the human variable region, constant region, and diversity region is introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. For example, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., as described supra. Monoclonal antibodies directed against the antigen are obtained from the immunized, transgenic mice using conventional hybridoma technology or plasmacytoma technology or any other method known in the art. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

### Antibody Fragments

As discussed supra antibody fragments are contemplated by the present invention. The term “antibody fragment” refers to a portion of a full-length antibody, generally the antigen binding or variable region. Examples of antibody fragments include Fab, Fab′, F(ab′)₂ and Fv fragments.

Papain digestion of an antibody produces two identical antigen binding fragments, called the Fab fragment, each with a single antigen binding site, and a residual “Fc” fragment.

Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, multispecific antibodies formed from antibody fragments, Fv fragments, F(ab) fragments and F(ab′)₂ fragments.

An “Fv” fragment is the minimum antibody fragment that contains a complete antigen recognition and binding site. This region consists of a dimer of one heavy and one light chain variable domain in a non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen.

A Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds. Springer-Verlag, New York, pp. 269-315 (1994).

### Antibody Isotypes

It will be appreciated by those skilled in the art that isotypes of the antibodies, antibody fragments and antibody conjugates can be switched to optimize clinical applications. For example, some isotypes (such as IgG2a) are superior effectors of antibody dependent cell cytotoxicity reactions. Likewise, some isotypes (such as IgG2a) are more readily eliminated from the circulation via the Fc receptors present on cells of the reticuloendothelial system and sequestered in the spleen than others, and, if bound to a target cell (which, for example, may be an effector of autoimmune disease), would be more efficient at removing the target cell from sites of active disease. Accordingly, certain antibody isotypes may be preferable to others, depending on the intended clinical application.

### Antibody Characterization

The present invention also provides a variable region of at least one complementarity determining region (CDR) of a V_{H} and/or V_{L} of a therapeutic antibody described herein according to any embodiment.

In this respect, the skilled artisan will be aware that each variable domain (e.g., a heavy chain V_{H} and/or light chain V_{L}) of an antibody comprises three hypervariable domains, sometimes called complementarity determining regions or “CDRs” flanked by four relatively conserved framework regions or “FRs”. A CDR contained in the variable domains of an antibody or antibody fragment is readily determined by a method described herein and/or known in the art, e.g., as described in Kabat, Sequences of Proteins of Immunological Interest (U.S. Department of Health and Human Services, third edition, 1983, fourth edition, 1987, fifth edition 1990). The person skilled in the art will readily appreciate that the variable domain of the antibody having the above-described variable domain is useful for the construction of other polypeptides or antibodies of desired specificity and biological function. Thus, the present invention also encompasses polypeptides and antibodies comprising at least one CDR of a variable domain of an antibody described herein according to any embodiment and/or which comprises at least one variable domain of an antibody as described according to any embodiment hereof.

In one example, nucleic acid encoding a desired monoclonal antibody may be readily isolated and sequenced using conventional procedures (e.g., by using oligonucleotide probes that are capable of binding specifically to genes encoding the heavy and light chains of an antibody, e.g., a murine antibody). For example, nucleic acid is isolated from a hybridoma cell expressing a monoclonal antibody. For example, RNA is isolated from hybridoma cells or from another transformed cell by standard techniques, such as guanidinium isothiocyanate extraction and precipitation followed by centrifugation or chromatography. Where desirable, mRNA is isolated from total RNA by standard techniques such as chromatography on oligo dT cellulose. Suitable techniques are described, for example, in Sambrook, Fritsch & Maniatis, whole of Vols I, II, and III. cDNA encoding a light chain and/or heavy chain of an antibody is then produced, either simultaneously or separately, using reverse transcriptase and DNA polymerase in accordance with methods known in the art. For example, total RNA is then converted to single stranded (ss) cDNA by reverse transcriptase using non-specific (oligo-dT or random hexamers) or specific (immunoglobulin CH1 or CK or Clambda constant region) oligonucleotide primers. Single-stranded cDNA produced by this reaction is amplified using the polymerase chain reaction in which ss cDNA, together with deoxynucleotide triphosphates, a DNA polymerase. (e.g., a thermostable polymerase) and specific primers, are used to amplify heavy or light chain variable region immunoglobulin genes. For example, using oligonucleotide primers capable of binding to consensus regions of constant regions of an antibody that flank or are adjacent to a variable domain of the antibody. These consensus regions are readily determined based on published heavy and light chain nucleic acid and/or amino acid sequences. Alternatively, suitable primers are described in U.S. Pat. No. 5,658,570. The primer used is, for example, a single stranded synthetic oligonucleotides ranging from 20 to 40 bases, containing some degenerate bases which bind to am immunoglobulin 5′ leader sequence. Optionally, a primer incorporates a restriction endonuclease cleavage site to permit directional cloning of PCR-amplified DNA into an appropriate expression vector possessing the same restriction site.

A suitable method for isolating nucleic acid encoding a variable domain of an antibody is described, for example, in U.S. Pat. No. 5,658,570.

In another example, lymphocytes producing an antibody of interest are selected by micromanipulation and nucleic acid encoding one or more variable domains isolated. For example, peripheral blood mononuclear cells are isolated from an immunized mammal and cultured for about 7 days in vitro. The cultures are screened for specific immunoglobulins capable of binding to a target antigen of interest. Cells producing immunoglobulin of interest are then isolated, e.g., by fluorescence activated cell sorting (FACS) or by identifying cells in a complement-mediated hemolytic plaque assay. Isolated cells are then micromanipulated into a suitable container and a nucleic acid encoding a V_{H} and/or V_{L} region is then isolated, e.g., using RT-PCR.

In a further example, nucleic acid sequences encoding a suitable useful for producing V_{H} and/or V_{L} region is obtained from a library of variable gene sequences from an animal of choice. For example, a library expressing random combinations of domains, e.g., V_{H} and/or V_{L} domains, is screened with a desired antigen to identify one or more library members capable of binding to the antigen with the desired affinity. Suitable libraries and methods for screening those libraries are known in the art and/or described in Huse et al. Science, 2476:1275, 1989; Francisco et al. PNAS, 90:10444, 1994; EP 368 684; and U.S. Pat. No. 5,969,108

The person skilled in the art will readily appreciate that a variable domains and/or a CDR isolated by a method described herein and/or known in the art is useful for producing a recombinant antibody according to a method known in the art and described in, for example, EP 0 451 216 or EP 0 549 581. Furthermore, the skilled artisan will be aware that binding affinity of a recombinant antibody and/or a variable domain and/or a CDR may be enhanced by making amino acid substitutions within a CDR or within a hypervariable loop or an antibody (see, for example, Chothia and Lesk, J. Mol. Biol. 196 901-917, 1987). Thus, the present invention also relates to antibodies wherein one or more of the mentioned CDRs comprise one or more, preferably not more than two amino acid substitutions.

### Antibody Conjugates

This invention also provides antibody conjugates comprising an antibody or antibody fragment conjugated or otherwise linked to a moiety such as a therapeutic agent e.g., a radioactive molecule, a toxin (e.g., calicheamicin), or a chemotherapeutic molecule, or to liposomes or other vesicles containing chemotherapeutic compounds. The antibody conjugates, when administered to an individual, can target these agents to a GAS cell recognized by the antibody or antibody fragment or other immunogenic moiety and thus can, for example, eliminate GAS cells and/or suppress proliferation and/or growth of GAS cells. With these methods, conjugation generally refers to linking these components as described herein. The linking (which is generally fixing these components in proximate association at least for administration) can be achieved in any number of ways, as described below.

A radioactive molecule includes any radioisotope that is effective in destroying a GAS. Examples include, but not limited to, cobalt-60 and X-rays. Additionally, naturally occurring radioactive elements such as uranium, radium, and thorium that typically represent mixtures of radioisotopes, are suitable examples of a radioactive molecule.

A toxin include, but not limited to, taxol, cytochalasin B, gramicidin D, ethidium bromide, emetine, mitomycin, etoposide, tenoposide, vincristine, vinblastine, Colchicin, doxorubicin, daunorubicin, dihydroxy anthracin diene, mitoxantrone, mithramycin, actinomycin D, 1-dehydrotestosterone, glucocorticoids, procaine, tetracaine, lidocaine, propranolol, and puromycin and analogs or homologs thereof.

The antibody may be linked to the radioactive molecule, the toxin, or the therapeutic molecule at any location along the antibody so long as the antibody is able to bind its target.

A toxin or a therapeutic agent may be coupled (e.g., covalently bonded) to a suitable monoclonal antibody or fragment thereof either directly or indirectly (e.g., via a linker group, or, alternatively, via a linking molecule with appropriate attachment sites, such as a platform molecule as described in U.S. Pat. No. 5,552,391). The toxin and therapeutic agent can be coupled directly to the particular targeting proteins using methods known in the art. For example, a direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

The antibodies can also be linked to a therapeutic agent via a microcarrier. Microcarrier is a biodegradable or a nonbiodegradable particle which is insoluble in water and which has a size of less than about 150, 120 or 100 mm in size, more commonly less than about 50-60 μm, preferably less than about 10, 5, 2.5, 2 or 1.5 μm. Microcarriers include “nanocarriers”, which are microcarriers have a size of less than about 1 μm, preferably less than about 500 nm. Such particles are known in the art. Solid phase microcarriers may be particles formed from biocompatible naturally-occurring polymers, synthetic polymers or synthetic copolymers, which may include or exclude microcarriers formed from agarose or cross-linked agarose, as well as other biodegradable materials known in the art. Biodegradable solid phase microcarriers may be formed from polymers which are degradable (e.g., poly(lactic acid), poly(glycolic acid) and copolymers thereof) or erodible poly(ortho esters such as 3,9-diethylidene-2,4,8,10-tetraoxaspiro[5.5]undecane (DETOSU) or poly(anhydrides), such as poly(anhydrides) of sebacic acid) under mammalian physiological conditions. Microcarriers may also be liquid phase (e.g., oil or lipid based), such liposomes, iscoms (immune-stimulating complexes, which are stable complexes of cholesterol, and phospholipid, adjuvant-active saponin) without antigen, or droplets or micelles found in oil-in-water or water-in-oil emulsions, provided the liquid phase microcarriers are biodegradable. Biodegradable liquid phase microcarriers typically incorporate a biodegradable oil, a number of which are known in the art, including squalene and vegetable oils. Microcarriers are typically spherical in shape, but microcarriers that deviate from spherical shape are also acceptable (e.g., ellipsoid, rod-shaped, etc.). Due to their insoluble nature with respect to water), microcarriers are filterable from water and water-based (aqueous) solutions.

The antibody conjugates may include a bifunctional linker that contains both a group capable of coupling to a toxic agent or therapeutic agent and a group capable of coupling to the antibody. A linker can function as a spacer to distance an antibody from an agent in order to avoid interference with binding capabilities. A linker can be cleavable or non-cleavable. A linker can also serve to increase the chemical reactivity of a substituent on an agent or an antibody, and thus increase the coupling efficiency. An increase in chemical reactivity may also facilitate the use of agents, or functional groups on agents, which otherwise would not be possible. The bifunctional linker can be coupled to the antibody by means that are known in the art. For example, a linker containing an active ester moiety, such as an N-hydroxysuccinimide ester, can be used for coupling to lysine residues in the antibody via an amide linkage. In another example, a linker containing a nucleophilic amine or hydrazine residue can be coupled to aldehyde groups produced by glycolytic oxidation of antibody carbohydrate residues. In addition to these direct methods of coupling, the linker can be indirectly coupled to the antibody by means of an intermediate carrier such as an aminodextran. In these embodiments the modified linkage is via either lysine, carbohydrate, or an intermediate carrier. In one embodiment, the linker is coupled site-selectively to free thiol residues in the protein. Moieties that are suitable for selective coupling to thiol groups on proteins are well known in the art. Examples include disulfide compounds, α-halocarbonyl and α-halocarboxyl compounds, and maleimides. When a micleophilic amine function is present in the same molecule as an α-halocarbonyl or carboxyl group the potential exists for cyclization to occur via intramolecular alkylation of the amine. Methods to prevent this problem are known to one of ordinary skill in the art, for example by preparation of molecules in which the amine and α-halo functions are separated by inflexible groups, such as aryl groups or trans-alkenes, that make the undesired cyclization stereochemically disfavored. See, for example, U.S. Pat. No. 6,441,163 for preparation of conjugates of maytansinoids and antibody via a disulfide moiety.

### Pharmaceutical Compositions

The present invention provides a pharmaceutical composition comprising a therapeutic antibody, antibody fragment or antibody conjugate or a derivative, analog or homolog and/or combinations thereof, including other active molecules and one or more pharmaceutically acceptable carriers and/or diluents. The active ingredients of a pharmaceutical composition comprising an antibody are contemplated herein to exhibit excellent therapeutic activity, for example, in the passive transfer of therapeutic antibodies to M protein of streptococci but said antibodies being only minimally reactive with heart tissue when administered in amount which depends on the particular case. For example, from about 0.1 ug to about 100 mg per kilogram of body weight per day may be administered. Dosage regima may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intranasal, intradermal or suppository routes or implanting (eg using slow release molecules). Depending on the route of administration, the active ingredients which comprise an antibody, antibody fragment or antibody conjugate may be required to be coated in a material to protect said ingredients from the action of enzymes, acids and other natural conditions which may inactivate said ingredients.

The active compounds may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

Sustained release injectable formulations are produced e.g., by encapsulating the antibody in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

When the therapeutic antibodies are suitably protected as described above, the active, compound may be orally administered, for example, with an inert diluent or with an assimilatable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 ug and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is known in the art. Except insofar as any conventional media or agent is incompatible with the active ingredient, use thereof in the therapeutic compositions is contemplated. Supplementary active ingredients can also be incorporated into the compositions.

It is advantageous to formulate parenteral compositions in dosage unit form for ease of administration and uniformity of dosage. “Dosage unit form” as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of a p-harmaceutical composition of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient is compounded for convenient and effective administration in effective amounts with a suitable pharmaceutically acceptable carrier in dosage unit form as hereinbefore disclosed. A unit dosage form can, for example, contain the principal active compound in an amount ranging from about 0.1 μg to about 100 mg. In the case of compositions containing supplementary active ingredients, the dosages are determined by reference to the usual dose and manner of administration of the said ingredients.

### Additional Components in Pharmaceutical Compositions

In another example, an antibody, antibody fragment or antibody conjugate that binds to an epitope of an M-protein of GAS is formulated in combination with another antimicrobial agent or antibiotic active against GAS. Combinations of the antibody, antibody fragment, antibody conjugate and other agents are useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics that may be combined with the antibody, antibody fragment or antibody conjugate include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

Therapy of Gas and/or Complications Thereof.

The compositions described according to any embodiment hereof may be administered to a subject to prevent severe GAS infection in a subject.

Accordingly, the present invention also encompasses methods for achieving a serum titer of at least about 40 μg/ml of one or more antibodies or fragments thereof that immunospecifically bind to one or more B-cell epitopes in a mammal, preferably a primate and most preferably a human. For example, the present invention provides methods for achieving a serum titer of at least about 40 μg/ml (preferably at least about 75 μg/ml, more preferably at least about 100 μg/ml, and most preferably at least about 150 μg/ml) of an antibody or fragment thereof that immunospecifically binds to a B-cell epitopes of M-protein GAS in a mammal, comprising administering a dose of less than 2.5 mg/kg (preferably 1.5 mg/kg or less) of the antibody to the non-primate mammal and measuring the serum titer of the antibody or antibody fragment at least 1 day after administering the dose to the mammal. The present invention also provides methods for achieving a serum titer of at least about 150 μg/ml (preferably at least about 200 μg/ml) of an antibody or fragment thereof that immunospecifically binds to a B-cell epitopes of M-protein GAS in a mammal, comprising administering a dose of approximately 5 mg/kg of the antibody or antibody fragment to the mammal and measuring the serum titer of the antibody or antibody fragment at least 1 day after the administration of the dose to the mammal.

The present invention also provides methods for achieving a serum titer of at about least 40 μg/ml of an antibody or fragment thereof that immunospecifically binds to a B-cell epitopes of M-protein GAS in a primate, comprising administering a first dose of 10 mg/kg (preferably 5 mg/kg or less and more preferably 1.5 mg/kg or less) of the antibody or antibody fragment to the primate and measuring the serum titer of the antibody or antibody fragment 20 days (preferably 25, 30, 35 or 40 days) after administrating the first dose to the primate and prior to the administration of any subsequent dose. The present invention also provides methods for achieving a serum titer of at least about 75 μg/ml (preferably at least about 100 μg/ml, at least about 150 μg/ml, or at least about 200 μg/ml) of an antibody or fragment thereof that immunospecifically binds to a B-cell epitopes of M-protein GAS in a primate, comprising administering a first dose of approximately 15 mg/kg of the antibody or antibody fragment to the primate and measuring the serum titer of the antibody or antibody fragment 20 days (preferably 25, 30, 35 or 40 days) after administering the first dose to the primate but prior to any subsequent dose.

The present invention also provides methods for preventing, treating, or ameliorating one or more symptoms associated with a GAS infection in a human subject, said methods comprising administering to said human subject at least a first dose of approximately 15 mg/kg of an antibody or fragment thereof that immunospecifically binds to a B-cell epitopes of M-protein GAS so that said human subject has a serum antibody titer of at least about 75 μg/ml, preferably at least about 100 μg/ml, at least about 150 μg/ml, or at least about 200 μg/ml 30 days after the administration of the first dose of the antibody or antibody fragment and prior to the administration of a subsequent dose. The present invention also provides methods for preventing, treating or ameliorating one or more symptoms associated with a GAS infection in a human subject, said methods comprising administering to said human subject at least a first dose of less than 15 mg/kg (preferably 10 mg/kg or less, more preferably 5 mg/kg or less, and most preferably 1.5 mg/kg or less) of an antibody or fragment thereof that immunospecifically binds to a B-cell epitopes of M-protein GAS so that said human subject has a serum antibody titer of at least about 75 μg/ml, preferably at least about 100 μg/ml, at least about 150 μg/ml, or at least about 200 μg/ml 30 days after the administration of the first dose of the antibody or antibody fragment and prior to the administration of a subsequent dose. The present invention further provides methods for preventing, treating or ameliorating one or more symptoms associated with a GAS infection in a human subject, said methods comprising administering to said human subject a first dose of an antibody or fragment thereof that immunospecifically binds to a B-cell epitopes of M-protein GAS such that a prophylactically or therapeutically effective serum titer of less than about 10 μg/ml is achieved no more than 30 days after administering the antibody or antibody fragment.

The present invention will now be illustrated by the following examples, which are not intended to be limiting in any way. The teachings of all references cited herein are incorporated herein by reference.

### Example 1

### Peptide Synthesis and Conjugation to a Peptide Carrier

The peptide J8 was synthesised and purified using high-performance liquid chromatography as described previously. Peptide was conjugated via a C-terminal cysteine residue to Diptheria toxoid (DT) (CSL), using 6′-maleimido-caproyl n-hydroxy succinimide (MCS), as described by Coligan et. al. The sequence for J8 peptide is QAEDKVKQSREAKKQVEKALKQLEDKVQ.

### Example 2

### Immunization of Mice

Inbred BALB/c and SCID mice (female, 4-6 weeks old) were obtained from Animal Resource Centre, Australia. Cohort of 10 BALB/c mice were immunised subcutaneously at the tail base on day 0 with 30 μg of J8-DT or DT in alum. Control mice received phosphate buffered saline (PBS/alum). All the groups also received three subsequent boosts on day 21, 28 and 35. Serum samples were collected on day 20, 27, 34 and 41 and IgG concentration measured by ELISA.

### Example 3

### Preparation of J8-DT Immune Serum

One week after the last boost (day 42) when the antibody titres were in the order of 10⁶, the mice were bled by cardiac puncture. The blood was allowed to clot at room temperature for 30 minutes followed by overnight storage at 4° C. for clot to retract. The clot was removed and supernatant was spun at 3000 rpm for 10 minutes. The serum after spinning was collected and stored at 4° C. until used.

### Example 4

### Passive Transfer of Immune Serum and GAS Challenge

The pooled serum (from each group) was transferred intra-peritoneally (ip) into the Balb/c and SCID mice in three doses of 0.5 ml each on day −1, 0 and +1 relative to the day of challenge. Two hours after the transfer of immune serum on day 0, the mice were tail bled and serum sample collected to measure IgG levels in the recipient mice. On day 0 the recipient mice were challenged intraperitoneally with a lethal dose of previously passaged M1 GAS as described previously. Following challenge, the mice were closely observed and their survival monitored on a day-to-day basis for 10 days.

### Example 5

### J8-DT Specific Antibody Production in Rabbits and Purification of Rabbit IgG

Antibodies in rabbits were raised at IMVS, Adelaide, S. A. Two rabbits were multiply vaccinated using 0.5 mg of J8-DT and DT antigen preparation in alum each. Four subsequent boosts were given at monthly intervals and serum samples were collected to quantitate antibodies to J8.

### Example 6

### Purification and Passive Transfer of Rabbit IgG

Rabbit IgG were purified using protein-G sepharose column. Briefly, rabbit antisera were diluted 1:2 and passed through protein G column for antibodies to bind to the column. The antibodies were then eluted using Glycine-HCl buffer pH (2.7), neutralized using Tris-HCl (pH 9), dialysed and concentrated. The purified IgG were administered intraperitoneally into mice on three consecutive days (day −1, 0 and +1) and challenged with M1 GAS on day 0.

### Example 7

### Antibody Assay/ELISA

ELISAs were performed for antibody determination. NUNC immunoplates (Flow laboratories) were coated with 100 μl of 5 μg/mL of Ag, as initially standardised in our laboratory, in carbonate-bicarbonate buffer, pH 9.6, overnight at 4° C. Rest of the procedure was similar to what has been described previously.

### Example 8

### In vivo CD4⁺ and CD8⁺ T cell Depletion

One week after the last inoculation of J8-DT/alum, mice in each group received 0.3 mg of rat anti-CD4 (GK1.5) mAb intraperitoneally (i.p.) before challenge and then twice per week for two weeks during the course of infection. Control mice included J8-DT immunised mice, which were treated with normal rat IgG (nRIg), and unimmunised mice which were treated with anti-CD4 mAb or nRIg. The dose and time-course for CD4 depletion was optimised beforehand using FACS to determine the degree of cell depletion. A rat IgG2a mAb clone 53.5.8 was used to grow anti-CD8 monoclonal antibodies which were then purified using protein-G column. Dose and time-course optimisation was then conducted and mice in immunised and control groups were injected with anti-CD8 mAb or nRIg. Data not shown.

### Example 9

### Passive Transfer of J8-DT Antisera in BALB/c Mice

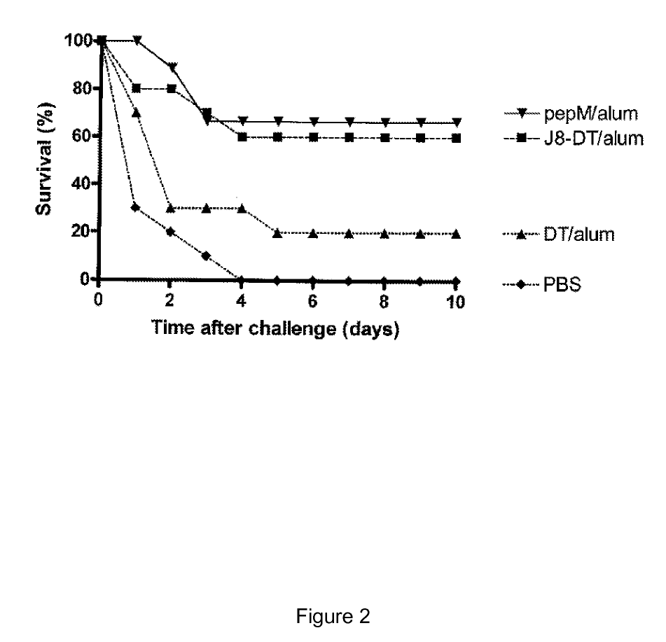

To determine the protective efficacy of antibody component of the serum, antisera was transferred into naïve animals, which were subsequently challenged with GAS M1 strain (FIG. 2). The results were compared using Mann-Whitney test. Animals receiving J8-DT antiserum survived significantly better than animals receiving control PBS antiserum (p<0.05). There was no significant difference between the level of protection conferred on naïve mice by sera from the PBS and DT immunised groups. Passive transfer of J8-DT antisera into BALB/c mice resulted in significantly higher number of survivors compared to mice that received DT antiserum. However, the vaccinated (J8-DT) challenged group were protected at a significantly higher level compared to the group that received J8-DT antiserum and then challenged. This observation indicated the possibility of involvement of other factors of immune system in vaccine mediated protection.

### Example 10

### Passive Transfer of J8-DT Anti Sera in SCID Mice

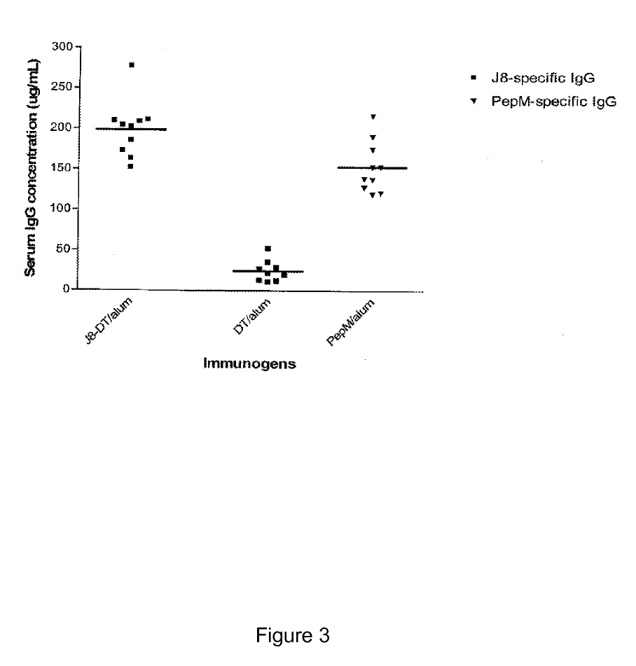

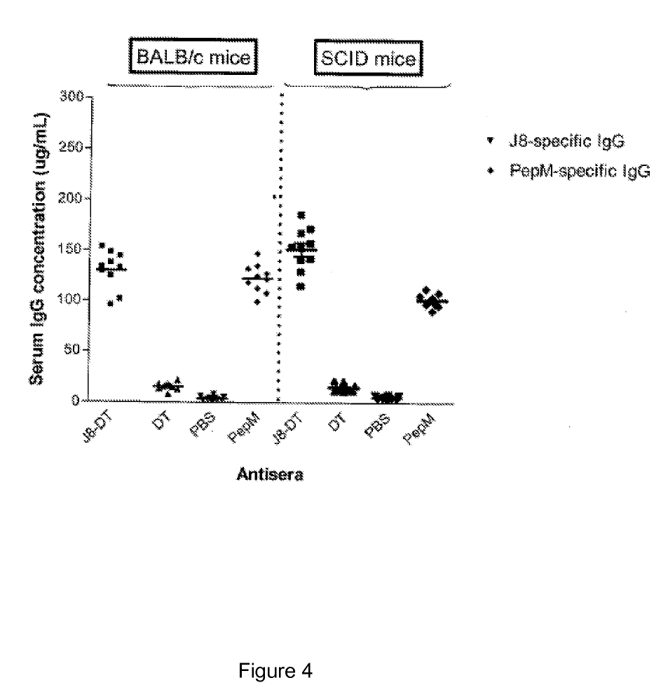

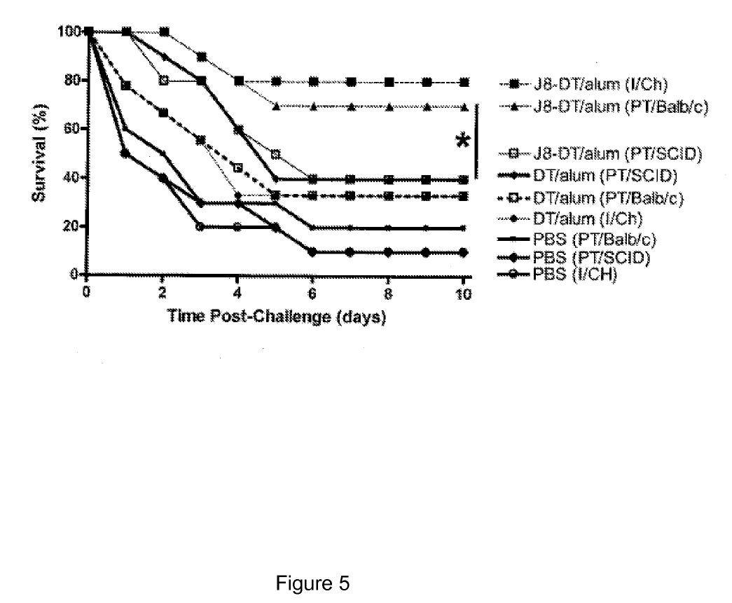

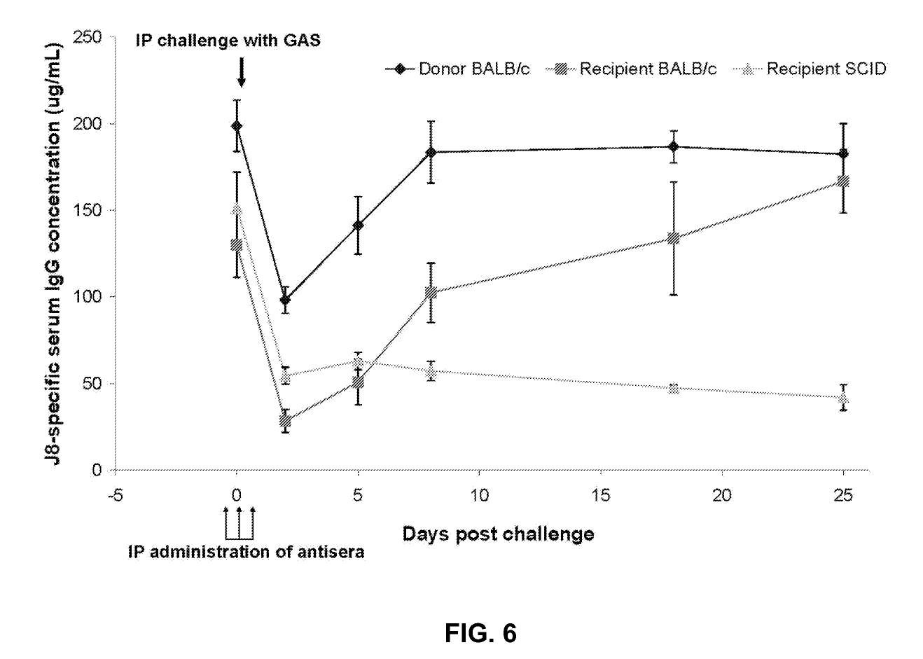

In order to exclude the possibility of involvement of host's immune components in J8-DT mediated protection, passive transfer studies were repeated in immunocompromised SCID mice which lack both B and T-cells (FIGS. 3-6). Our results demonstrated that there was significant difference in survival between BALB/c and SCID J8-DT antiserum recipient mice. This reflects to the possibility that once the passively transferred antibodies were worn out, SCID mice due to their inability to mount a specific immune response against GAS were not protected. This was also confirmed by ELISA results that demonstrated that majority of the antibodies were consumed both in BALB/c and SCID mice soon after bacterial challenge. Since BALB/c mice were able to continue synthesizing adequate levels of IgG (as demonstrated by ELISA), survived (p<0.05) whereas SCID mice due to absence of protective antibodies succumbed to infection.

### Example 11

### Transfer of Purified Rabbit IgG in Mouse and Assessment of Protection

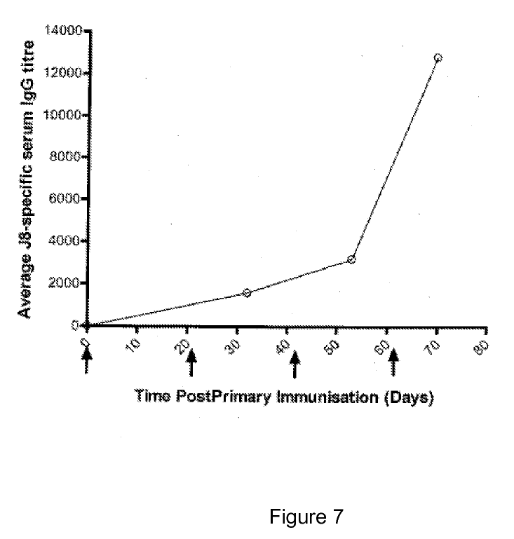

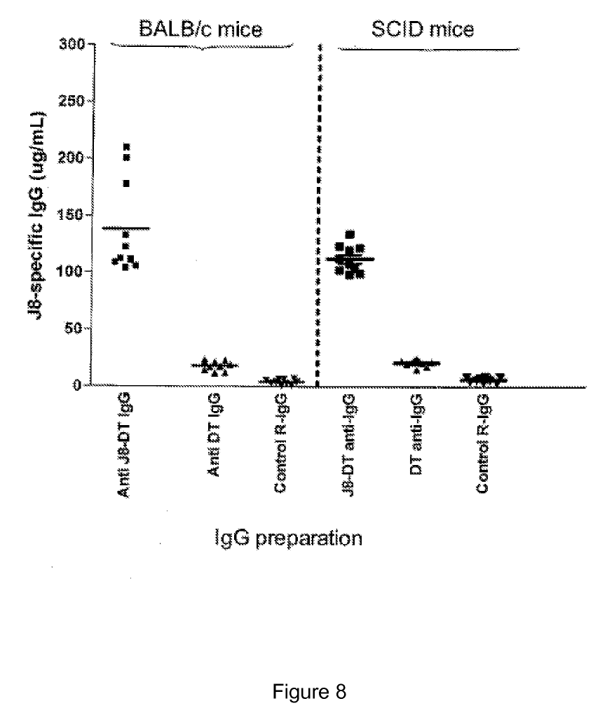

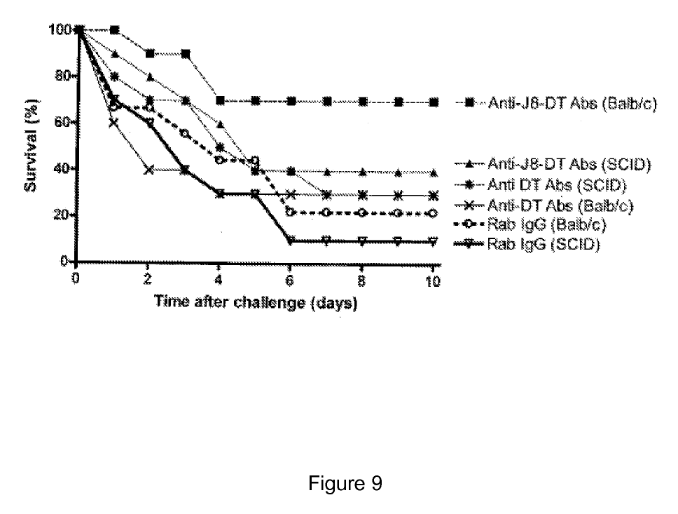

To demonstrate that the protective factors in serum are IgG antibodies, we passively transferred affinity purified rabbit IgG into BALB/c and SCID mice (FIGS. 7-10). Rabbits were immunised subcutaneously with J8-DT/alum on days 0, 21, 42 and 63 post-primary immunisation. Following the final boost rabbit antisera was collected.

IgG was purified from the antisera collected from the immunised rabbits using a Protein G column. The purified IgG was then administered intraperitoneally to naive recipient BALB/c and SCID mice in 3×0.5 ml doses on day −1, O and +1. On day 0 mice were challenged with GAS.

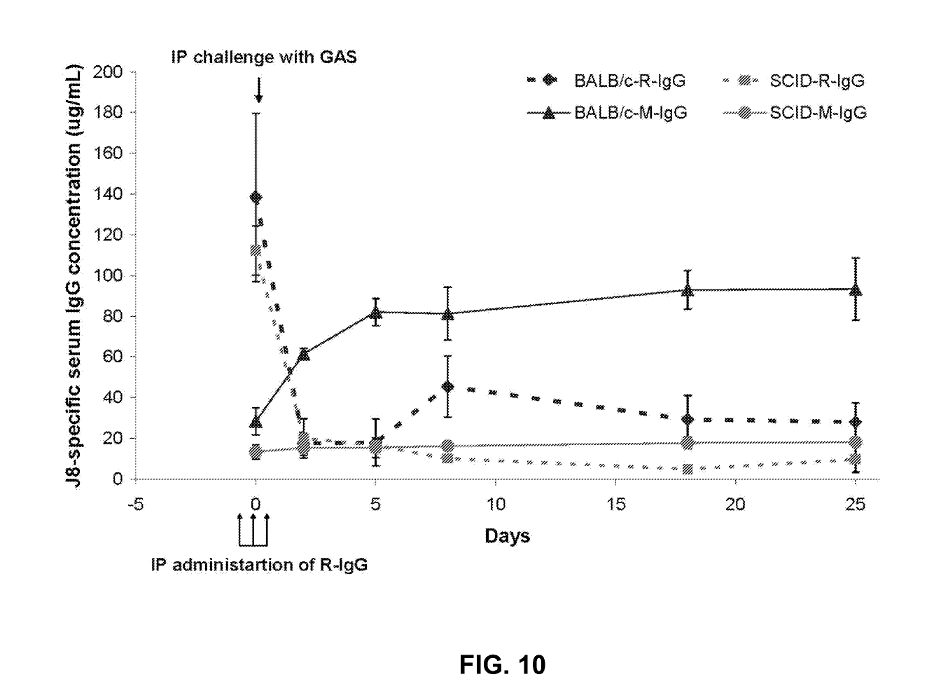

Following intraperitoneal GAS challenge the recipient BALB/c and SCID mice were bled to determine the level of J8-specific rabbit IgG. J8-specific rabbit IgG is rapidly consumed following GAS infection (FIG. 10). Moreover, the level of J8-specific mouse IgG was also determined. The J8-specific mouse IgG in BALB/c mice is generated following GAS challenge as a response to the bacterial infection, in contrast, SCID mice are incapable of generating an antibody response to the pathogen.

### Example 12

### Role of CD4+ T Cells in Protection Against GAS Challenge

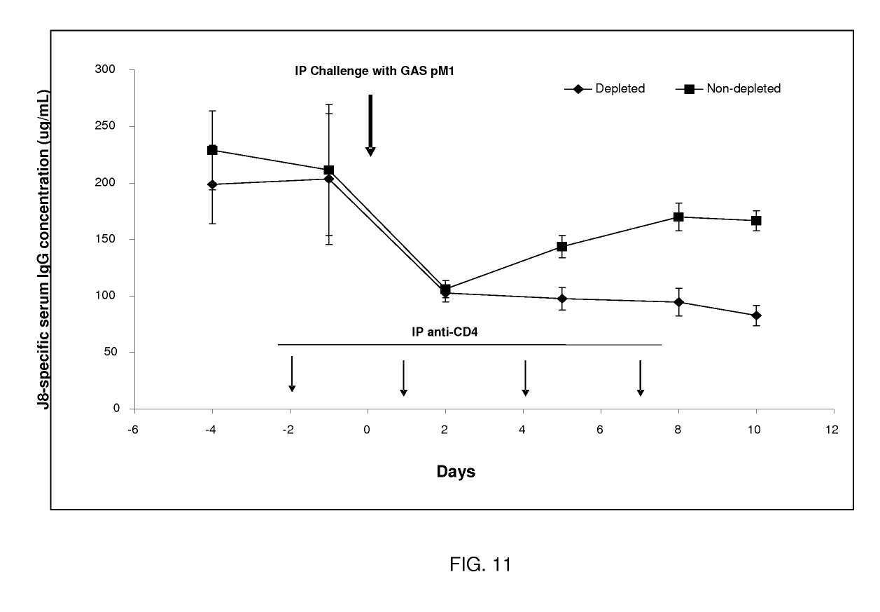

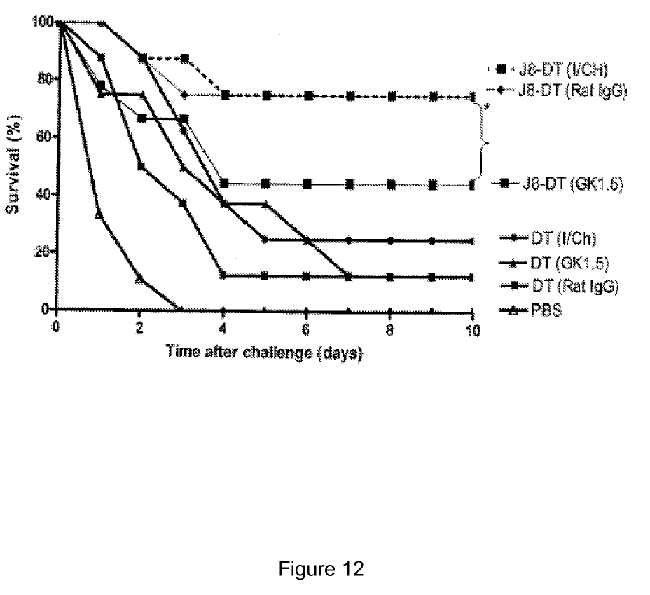

BALB/c mice were immunised with J8-DT, DT or PBS as previously described. One week after the final boost the mice were depleted of their CD4+ T-cell population using GK1.5 anti-CD4 antibodies. Antibody concentrations (FIG. 11) and survival curve (FIG. 12) in immunised and CD4+ T-cell depleted/non-depleted mice before and after challenge are shown. A correlation between antibody levels post challenge and survival is observed. The abbreviation I/Ch stands for immunised/challenged mice (positive controls).

### Example 13

### Therapeutic Treatment of GAS Infection

BALB/c or SCID mice were infected with GAS essentially as described herein, and treated with mouse anti-J8-DT antiserum produced essentially as described in Example 3. Treatment groups were as follows:

| Donor mice | Antiserum recipient | Antiserum recipient |
|---|---|---|
| (BALB/c) | mice (BALB/c) | mice (SCID) |
| J8-DT(I/Ch)- | J8-DT | J8-DT |
| Positive control | | |
| J8-DT(PT/Ch)- | DT | DT |
| (control) | | |
| PBS(I/Ch) | PBS | PBS |
| (Negative control) | | |

I/Ch: Mice immunised with antigens and challenged with GAS
PT/Ch: Naïve mice administered with antisera before and after GAS challenge
T: Naïve mice treated with antisera post-challenge

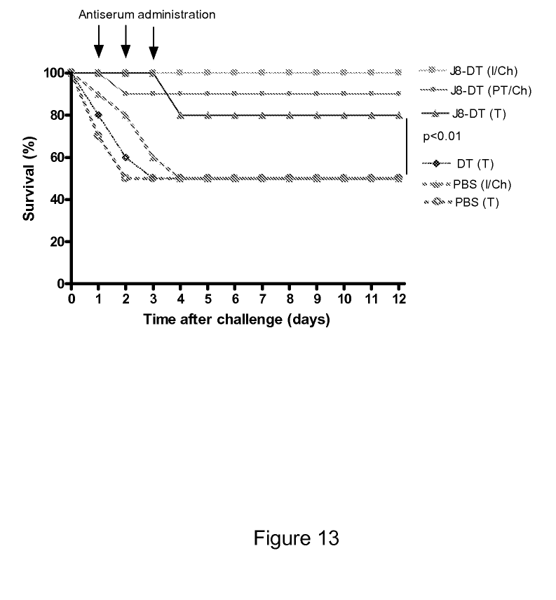

Briefly, mice were infected with GAS at day 0, and treated with antiserum four hours later and again 1 days later and 2 days later. For SCID mice an additional dose of antiserum was administered 3 days following infection. As shown in FIG. 13, the survival rate BALB/c mice treated with antiserum following infection is similar to that achieved with mice immunized with the J8-DT peptide or treated with antiserum prior to and after infection. Moreover, the survival rate of mice treated with antiserum following infection is significantly higher than the survival rate of negative controls (p<0.01).

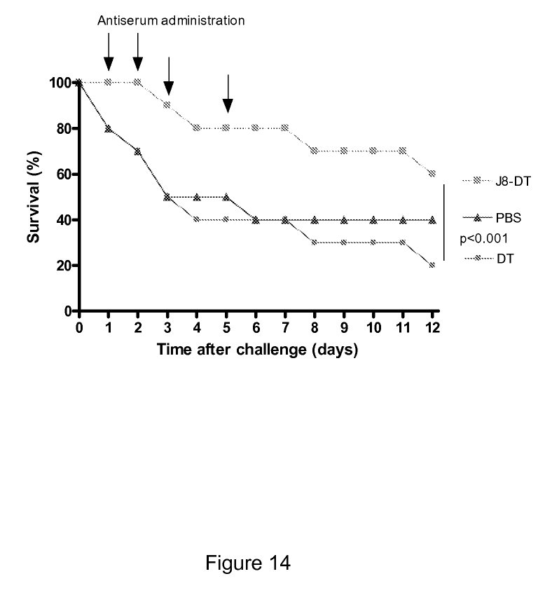

Similarly, the survival rate of SCID mice, which lack both B cells and T cells treated with antiserum following infection is significantly higher than the survival rate of negative control populations (FIG. 14).

### Example 14

### Monoclonal Antibodies Against GAS M Protein

14.1 Monoclonal Antibodies that Bind GAS M Protein

Peptides comprising a sequence set forth in one of SEQ ID NOs: 2-10 or 14-24 or 26 are produced synthetically. The peptides are then suspended in PBS (Phosphate Buffered Saline) to obtain an antigen solution.

14.2 Immunization of Mice with Antigen

The antigen solution is then mixed with Freund's complete adjuvant (manufactured by DIFCO) in a 1:1 ratio and the mixture emulsified. The emulsion is then injected into female Balb/C mice (8-week old, about 100 μg/mouse of peptide) subcutaneously.

Booster immunizations of an emulsified mixture of the appropriate antigen solution (about 100 μg/mouse of peptide) and Freund's incomplete adjuvant (manufactured by DIFCO) (in a 1:1 ratio) are administered by injection at about 2 week intervals by subcutaneous injection. Three days after the third booster, a blood sample is collected from the tail vein and the antibody titer in the serum is measured by a direct solid phase ELISA. Briefly, the antigen solution is diluted with PBS and the resulting solution adsorbed to an ELISA plate for approximately 2 hours. The plate is then blocked by a 4-fold dilution of Blockace (manufactured by Snow Brand Milk Products) in PBS. After washing the plate, various dilutions of the serum obtained from the immunized mice in a serum diluting buffer (PBS containing 5% FBS) are added to each well of the plate and incubated at room temperature for 2 hours. Following washing the plates a HRP conjugated rat anti-mouse immunoglobulin antibody (Clontech, Palo Alto, Calif., USA) diluted in diluent buffer (0.1% casein, 0.1% (v/v) Tween 20 and 0.1% (w/v) Thimersol in PBS) is added to each well of the plates. Plates are then incubated at room temperature for 1 hour on an agitator. TMB (3,3′,5′,5-Tetramethylbenzidine) Sigma Aldrich, Sydney, Australia) is then warmed to room temperature. Plates are then washed and TMB is added to each well and the plated incubated at room temperature (in the dark) for approximately 20-30 minutes. Reactions are stopped with 0.5M sulfuric acid. Absorbance is then read with a PowerWave_{X} 340 plate reader, Bio-Tek instruments Inc., Winooski, Vt.). Absorbance is detected at 450 nm and 620 nm. The level of absorbance detected in negative control wells (no peptide) is subtracted from the absorbance of each other well to determine the amount of antibody bound to GAS M protein peptide.

For those mice that have raised an immune response that is specific to the peptide with which they were immunized or GAS M protein, approximately two weeks after the third booster, a solution of the appropriate antigen solution for immunization in physiological saline is administered intraperitoneally. Three days after the administration, spleen cells are prepared from the immunized mice to produce cell fusions for the production of hybridomas.

### 14.3 Cell Fusion and Production of Hybridoma

Three days after the last immunization, spleens of immunized mice are excised prepared for cell fusion. Balb/c mouse-derived myeloma SP cells (which are a strain that lack hypoxanthine-guanine phosphoribosyl transferase (HGPRT)) are used as the parental strain for cell-fusion. SP2 cells (2×10⁷ cells) and spleen cells (1×10⁸ cells) are combined and cell fusion conducted according to conventional methods by using polyethylene glycol 4000 (PEG4000™, manufactured by Merck) as a cell fusion promoter.

Cells are suspended in a culture medium (HAT medium) and distributed into a 96-well plate. The fused cells are cultured at 37° C. supplemented with 5% CO₂. Media is changed every 3 to 5 days. Only those hybridomas that grow in the medium are selected and cultured further.

Culture medium from hybridomas are then screened using the ELISA assay described supra. Only those hybridomas that secrete monoclonal antibodies capable of binding to the relevant peptide are selected for cloning.

Those hybridomas that produce monoclonal antibodies capable of binding the antigen are cloned using limited dilution and the cells isolated and frozen

### 14.4 Preparation and Purification of Monoclonal Antibodies

Pristan (0.5 ml/mouse) is administered to female Balb/c mice (8-week old) intraperitoneally and, ten days after administration, a hybridoma (approximately 10⁷ cells/0.5 ml/mouse) is injected intraperitoneally. Ascites are collected by an 18G injection needle. The ascites collected is centrifuged at 1,000 r.p.m. at 4° C. for approximately 10 minutes and the supernatant allowed to stand at 37° C. for 30 minutes. Supernatant is then incubated at 4° C. overnight.

The supernatant is then centrifuged at 12,000 r.p.m. at 4° C. for 10 minutes, and the resultant supernatant applied to a Sephrose Protein A affinity column (manufactured by Pharmacia Bioteck) to purify the monoclonal antibody. The absorbance of a solution of the antibody is measured at 260, 280 and 320 nm and the antibody concentration is determined by Werbulg-Christian method.

### 14.5 Passive Immunization of Mice

Mice are treated with monoclonal antibodies either prior to or after challenge with GAS essentially as described in Examples 10 and 13. Survival rate of mice treated with antibodies are then determined, and antibodies that increase the rate of survival compared to mice that have not been treated are selected.

1. A composition comprising an amount of an antibody, antibody fragment or antibody conjugate sufficient to treat Group A streptococcus (GAS) infection or complication thereof in a subject or a disease or complication associated with GAS infection in a subject wherein said antibody, antibody fragment or antibody conjugate binds immunospecifically to a B-cell epitope of GAS M-protein.

2. The composition of claim 1, wherein the antibody, antibody fragment or antibody conjugate comprises a polyclonal antibody or immunoglobulin fraction or fragment thereof.

3. The composition of claim 1, wherein the antibody, antibody fragment or antibody conjugate comprises a monoclonal antibody or fragment thereof or a humanized derivative of said monoclonal antibody or said fragment that binds immunospecifically to a B-cell epitope of Group A streptococci (GAS) M-protein.

4. The composition of claim 1, wherein the antibody, antibody fragment or antibody conjugate comprises a recombinant antibody.

5. The composition of claim 1, wherein the antibody, antibody fragment or antibody conjugate comprises a humanized antibody or humanized antibody fragment.

6. The composition of claim 1, wherein the antibody, antibody fragment or antibody conjugate is reactive with a conformational epitope of an M-protein of S. pyogenes (GAS) and only minimally reactive or non-reactive with a tissue of a subject to whom the antibody is administered.

7. The composition of claim 6, wherein the conformational epitope of an M-protein of S. pyogenes (GAS) comprises the sequence REAK (SEQ ID NO:14).

8. The composition of claim 1, wherein the antibody, antibody fragment or immunogenic moiety of the antibody conjugate is modified to enhance stability in vivo thereby enhancing the effective serum titer of a unit dose of said antibody, antibody fragment or antibody conjugate.

9. (canceled)

10. (canceled)

11. A method of producing an antibody, antibody fragment or antibody conjugate, said method comprising immunizing an animal with an immunogenic peptide composition comprising a chimeric peptide comprising a first amino acid sequence comprising a conformational epitope of an M-protein of Streptococcus pyogenes (GAS) linked to or within a second amino acid sequence having the same native conformation as the first sequence for a time and under conditions to elicit antibody production, isolating serum from the animal and then producing an antibody, antibody fragment or antibody conjugate from the serum.

12. The method of claim 11, wherein the immunogenic peptide composition comprises a carrier protein.

13. The method of claim 11, wherein the conformational epitope of an M protein of S. pyogenes (GAS) is embedded in a heptad repeat sequence.

14. The method of claim 11, wherein the produced antibody, antibody fragment or antibody conjugate is reactive with a B cell epitope of an M-protein of S. pyogenes (GAS) and only minimally reactive with tissues of the animal.

15. (canceled)

16. A pharmaceutical composition comprising a unit dose of the composition of claim 1 and a pharmaceutically acceptable carrier or excipient.

17. A method for the prophylactic or therapeutic treatment of Group A streptococcus (GAS) infection or a complication thereof in a subject comprising administering an amount of the composition of claim 1 to the subject effective to prevent an increase in bacterial count or reduce bacterial count in a sample from the subject or reduce the severity of one or more disease symptoms or prevent onset of one or more diseases arising from GAS infection.

18. The method of claim 17, wherein the subject is a non-vaccinated subject that has not been vaccinated previously with a peptide-based vaccine comprising an immunogenic peptide derived from a protein of Streptococcus pyogenes.

19. The method of claim 17, wherein the subject is immune-compromized such that the subject does not produce endogenous antibody at a level sufficient to prevent the spread or development of GAS infection or the progression of disease arising from GAS infection.

20. The method of claim 17, wherein the subject is immunodeficient such that the subject does not have a functional immune system sufficient to produce endogenous antibody at a level to prevent the spread or development of GAS infection or the progression of disease arising from GAS infection.

21. The method of claim 17, wherein the administered composition comprises and amount of a polyclonal antiserum that binds immunospecifically to a B-cell epitope of GAS M-protein comprising the sequence set forth in SEQ ID NO: 9.

22. The method of claim 17, wherein the administered composition is formulated with a pharmaceutically acceptable carrier or excipient before administration to the subject.

23. The method of claim 17 wherein the composition is coadministered with an antibiotic having bacteriostatic or bacteriocidal activity against Streptococcus pyogenes (GAS).

24. A method of maintaining a therapeutically or prophylactically effective serum titer of an antibody against an M-protein of Group A streptococcus (GAS) in a subject comprising administering a plurality of doses of a composition according to claim 1 to a subject, wherein said each of said doses is administered in an amount effective to prevent an increase in bacterial count or to reduce bacterial count in a sample from the subject and/or to reduce the severity of one or more disease symptoms or to prevent onset of one or more diseases arising from GAS infection and/or to opsonize said pathogen in the serum of the subject.

25. (canceled)


SEQUENCE LISTING (27 sequences)

SEQ ID NO: 1 - protein, 10 aa, Artificial Sequence
  Description of Artificial Sequence Synthetic peptide
RDLDASREAK

SEQ ID NO: 2 - protein, 28 aa, Artificial Sequence
  Description of Artificial Sequence Synthetic peptide
QLEDKVKQLRRDLDASREAKEELQDKVK

SEQ ID NO: 3 - protein, 28 aa, Artificial Sequence
  Description of Artificial Sequence Synthetic peptide
LEDKVKQARRDLDASREAKKELQDKVKQ

SEQ ID NO: 4 - protein, 28 aa, Artificial Sequence
  Description of Artificial Sequence Synthetic peptide
EDKVKQAERDLDASREAKKQLQDKVKQL

SEQ ID NO: 5 - protein, 28 aa, Artificial Sequence
  Description of Artificial Sequence Synthetic peptide
DKVQKAEDDLDASREAKKQVQDKVKQLE

SEQ ID NO: 6 - protein, 28 aa, Artificial Sequence
  Description of Artificial Sequence Synthetic peptide
KVKQAEDKLDASREAKKQVEDKVKQLED

SEQ ID NO: 7 - protein, 28 aa, Artificial Sequence
  Description of Artificial Sequence Synthetic peptide
VKQAEDKVDASREAKKQVEKKVKQLEDK

SEQ ID NO: 8 - protein, 28 aa, Artificial Sequence
  Description of Artificial Sequence Synthetic peptide
KQAEDKVKASREAKKQVEKAVKQLEDKV

SEQ ID NO: 9 - protein, 28 aa, Artificial Sequence
  Description of Artificial Sequence Synthetic peptide
QAEDKVKQSREAKKQVEKALKQLEDKVQ

SEQ ID NO: 10 - protein, 28 aa, Artificial Sequence
  Description of Artificial Sequence Synthetic peptide
AEDKVKQLREAKKQVEKALEQLEDKVQL

SEQ ID NO: 11 - protein, 29 aa, Artificial Sequence
  Description of Artificial Sequence Synthetic peptide
KQAEDKVKASREAKKQVEKALEQLEDKVK

SEQ ID NO: 12 - protein, 20 aa, Artificial Sequence
  Description of Artificial Sequence Synthetic peptide
LRRDLDASREAKKQVEKALE

SEQ ID NO: 13 - protein, 7 aa, Artificial Sequence
  Description of Artificial Sequence Synthetic peptide
VKQLEDK

SEQ ID NO: 14 - protein, 4 aa, Artificial Sequence
  Description of Artificial Sequence Synthetic peptide
REAK

SEQ ID NO: 15 - protein, 12 aa, Artificial Sequence
  Description of Artificial Sequence Synthetic peptide
LRRDLDASREAK

SEQ ID NO: 16 - protein, 12 aa, Artificial Sequence
  Description of Artificial Sequence Synthetic peptide
RRDLDASREAKK

SEQ ID NO: 17 - protein, 12 aa, Artificial Sequence
  Description of Artificial Sequence Synthetic peptide
RDLDASREAKKQ

SEQ ID NO: 18 - protein, 12 aa, Artificial Sequence
  Description of Artificial Sequence Synthetic peptide
DLDASREAKKQV

SEQ ID NO: 19 - protein, 12 aa, Artificial Sequence
  Description of Artificial Sequence Synthetic peptide
LDASREAKKQVE

SEQ ID NO: 20 - protein, 12 aa, Artificial Sequence
  Description of Artificial Sequence Synthetic peptide
DASREAKKQVEK

SEQ ID NO: 21 - protein, 12 aa, Artificial Sequence
  Description of Artificial Sequence Synthetic peptide
ASREAKKQVEKA

SEQ ID NO: 22 - protein, 12 aa, Artificial Sequence
  Description of Artificial Sequence Synthetic peptide
SREAKKQVEKAL

SEQ ID NO: 23 - protein, 12 aa, Artificial Sequence
  Description of Artificial Sequence Synthetic peptide
REAKKQVEKALE

SEQ ID NO: 24 - protein, 14 aa, Artificial Sequence
  Description of Artificial Sequence Synthetic peptide
ASREAKKQVEKALE

SEQ ID NO: 25 - protein, 7 aa, Artificial Sequence
  Description of Artificial Sequence Synthetic peptide
VKQXEDX

SEQ ID NO: 26 - protein, 28 aa, Artificial Sequence
  Description of Artificial Sequence Synthetic peptide
DKVKQAEDKVKQLEDKVEELQDKVKQLE

SEQ ID NO: 27 - protein, 6 aa, Artificial Sequence
  Description of Artificial Sequence Synthetic 6xHis tag
HHHHHH